SEQUENCE LISTING (31 sequences)

SEQ ID NO: 1 - protein, 120 aa, synthetic construct
  REGION 1..120: note = PAN Domain consensus sequence
  VARIANT 2..4: note = Xaa can be any naturally occurring amino acid
  VARIANT 11..15: note = Xaa can be any naturally occurring amino acid
  VARIANT 22..24: note = Xaa can be any naturally occurring amino acid
  VARIANT 35..44: note = Xaa can be any naturally occurring amino acid
  VARIANT 46..48: note = Xaa can be any naturally occurring amino acid
  VARIANT 51..54: note = Xaa can be any naturally occurring amino acid
  VARIANT 56: note = Xaa can be any naturally occurring amino acid
  VARIANT 64: note = Xaa can be any naturally occurring amino acid
  VARIANT 67..79: note = Xaa can be any naturally occurring amino acid
  VARIANT 81: note = Xaa can be any naturally occurring amino acid
  VARIANT 90: note = Xaa can be any naturally occurring amino acid
  VARIANT 93..106: note = Xaa can be any naturally occurring amino acid
  VARIANT 109: note = Xaa can be any naturally occurring amino acid
  VARIANT 118..119: note = Xaa can be any naturally occurring amino acid
CXXXLIVMFYXXXXXLIVMFYXXXDENQLIVMFYXXXXXXXXXXCXXXCTXXXXCXLIVMFYFXFYXXXXXXXXXXXXXCXLIVMFYRKXSTXXXXXXXXXXXXXXSGXSTLIVMFYXXC

SEQ ID NO: 2 - protein, 125 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYI

SEQ ID NO: 3 - protein, 121 aa, synthetic construct
  REGION 1..121: note = PAN domain consensus sequence
  VARIANT 2..4: note = Xaa can be any naturally occurring amino acid
  VARIANT 11..15: note = Xaa can be any naturally occurring amino acid
  VARIANT 22..24: note = Xaa can be any naturally occurring amino acid
  VARIANT 35..44: note = Xaa can be any naturally occurring amino acid
  VARIANT 46..48: note = Xaa can be any naturally occurring amino acid
  VARIANT 51..54: note = Xaa can be any naturally occurring amino acid
  VARIANT 56: note = Xaa can be any naturally occurring amino acid
  VARIANT 64: note = Xaa can be any naturally occurring amino acid
  VARIANT 67..79: note = Xaa can be any naturally occurring amino acid
  VARIANT 81: note = Xaa can be any naturally occurring amino acid
  VARIANT 90: note = Xaa can be any naturally occurring amino acid
  VARIANT 93..107: note = Xaa can be any naturally occurring amino acid
  VARIANT 110: note = Xaa can be any naturally occurring amino acid
  VARIANT 119..120: note = Xaa can be any naturally occurring amino acid
CXXXLIVMFYXXXXXLIVMFYXXXDENQLIVMFYXXXXXXXXXXCXXXCTXXXXCXLIVMFYFXFYXXXXXXXXXXXXXCXLIVMFYRKXSTXXXXXXXXXXXXXXXSGXSTLIVMFYXXC

SEQ ID NO: 4 - protein, 121 aa, synthetic construct
  REGION 1..121: note = PAN domain consensus sequence
  VARIANT 2..4: note = Xaa can be any naturally occurring amino acid
  VARIANT 11..15: note = Xaa can be any naturally occurring amino acid
  VARIANT 22..24: note = Xaa can be any naturally occurring amino acid
  VARIANT 35..44: note = Xaa can be any naturally occurring amino acid
  VARIANT 46..48: note = Xaa can be any naturally occurring amino acid
  VARIANT 51..54: note = Xaa can be any naturally occurring amino acid
  VARIANT 56: note = Xaa can be any naturally occurring amino acid
  VARIANT 64: note = Xaa can be any naturally occurring amino acid
  VARIANT 67..80: note = Xaa can be any naturally occurring amino acid
  VARIANT 82: note = Xaa can be any naturally occurring amino acid
  VARIANT 91: note = Xaa can be any naturally occurring amino acid
  VARIANT 94..107: note = Xaa can be any naturally occurring amino acid
  VARIANT 110: note = Xaa can be any naturally occurring amino acid
  VARIANT 119..120: note = Xaa can be any naturally occurring amino acid
CXXXLIVMFYXXXXXLIVMFYXXXDENQLIVMFYXXXXXXXXXXCXXXCTXXXXCXLIVMFYFXFYXXXXXXXXXXXXXXCXLIVMFYRKXSTXXXXXXXXXXXXXXSGXSTLIVMFYXXC

SEQ ID NO: 5 - protein, 122 aa, synthetic construct
  REGION 1..122: note = PAN domain consensus sequence
  VARIANT 2..4: note = Xaa can be any naturally occurring amino acid
  VARIANT 11..15: note = Xaa can be any naturally occurring amino acid
  VARIANT 22..24: note = Xaa can be any naturally occurring amino acid
  VARIANT 35..44: note = Xaa can be any naturally occurring amino acid
  VARIANT 46..48: note = Xaa can be any naturally occurring amino acid
  VARIANT 51..54: note = Xaa can be any naturally occurring amino acid
  VARIANT 56: note = Xaa can be any naturally occurring amino acid
  VARIANT 64: note = Xaa can be any naturally occurring amino acid
  VARIANT 67..80: note = Xaa can be any naturally occurring amino acid
  VARIANT 82: note = Xaa can be any naturally occurring amino acid
  VARIANT 91: note = Xaa can be any naturally occurring amino acid
  VARIANT 94..108: note = Xaa can be any naturally occurring amino acid
  VARIANT 111: note = Xaa can be any naturally occurring amino acid
  VARIANT 120..121: note = Xaa can be any naturally occurring amino acid
CXXXLIVMFYXXXXXLIVMFYXXXDENQLIVMFYXXXXXXXXXXCXXXCTXXXXCXLIVMFYFXFYXXXXXXXXXXXXXXCXLIVMFYRKXSTXXXXXXXXXXXXXXXSGXSTLIVMFYXXC

SEQ ID NO: 6 - DNA, 25 bp, synthetic construct
  misc_feature 1..25: note = c-MET Forward Primer
  source 1..25: mol_type = other DNA
ttaaaggagacctcaccatgtaatc

SEQ ID NO: 7 - DNA, 22 bp, synthetic construct
  misc_feature 1..22: note = c-MET Reverse Primer
  source 1..22: mol_type = other DNA
cctgatcgagaaaccacaacct

SEQ ID NO: 8 - DNA, 25 bp, synthetic construct
  misc_feature 1..25: note = MMP9 Forward Primer
  source 1..25: mol_type = other DNA
gatccaaaactactcggaagacttg

SEQ ID NO: 9 - DNA, 15 bp, synthetic construct
  misc_feature 1..15: note = MMP9 Reverse Primer
  source 1..15: mol_type = other DNA
gaaggcgcgggcaaa

SEQ ID NO: 10 - DNA, 21 bp, synthetic construct
  misc_feature 1..21: note = GAPDH Forward Primer
  source 1..21: mol_type = other DNA
ttgccatcaatgaccccttca

SEQ ID NO: 11 - DNA, 19 bp, synthetic construct
  misc_feature 1..19: note = GAPDH Reverse Primer
  source 1..19: mol_type = other DNA
cgccccacttgattttgga

SEQ ID NO: 12 - DNA, 36 bp, synthetic construct
  misc_feature 1..36: note = AA340F Primer
  source 1..36: mol_type = other DNA
aacttgtactttcaaggctaccaggattcggtaaat

SEQ ID NO: 13 - DNA, 37 bp, synthetic construct
  misc_feature 1..37: note = RLK7_AA448R Primer
  source 1..37: mol_type = other DNA
acaagaaagctgggtcctacacttttaagaatgaagt

SEQ ID NO: 14 - DNA, 36 bp, synthetic construct
  misc_feature 1..36: note = RLK5_AA322F Primer
  source 1..36: mol_type = other DNA
aacttgtactttcaaggcgctctgaattgtgattcc

SEQ ID NO: 15 - DNA, 37 bp, synthetic construct
  misc_feature 1..37: note = RLK5_AA400R Primer
  source 1..37: mol_type = other DNA
acaagaaagctgggtcctaagatatcgagcttaaaga

SEQ ID NO: 16 - protein, 86 aa, Caenorhabditis elegans
SGEYAFTYLSDRYMDQKSVIKWTTKANLEECLSDCLDEKSFECRSISFNRTDGGCHMSKDSQISRPEAIRLNNNPNYRIDYYENNC

SEQ ID NO: 17 - protein, 84 aa, Drosophila melanogaster
PSRRYVFERHPRKKLKLPISDIKEITAANRSDCEDKCLNEFSFVCRSANFDSTMRSCTLSRFTRRTHPELMEDDPNSDYLENTC

SEQ ID NO: 18 - protein, 84 aa, Danio rerio
KEGRNFLVYYDSRLAGVPSNAIRRPINITTSTNVLNFCAAFCLRERACQAFSFTNTTTPSCFWVTSGVSQLSPSPQTFTYLKNT

SEQ ID NO: 19 - protein, 79 aa, Rattus norvegicus
SLDGYVSTQGASLHSLTKKQLAAGSIADCLAKCEGETDFICRSFQYHSKEQQCVIVMAENSKTSSIIRMRDVILFEKRV

SEQ ID NO: 20 - protein, 78 aa, Bos taurus
LLDDYVNTQGASLLSLSRKNLAGRSVEDCAAKCEEETDFVCRAFQYHSKEQQCVVMAENSKNTPVFRMRDVILYEKRI

SEQ ID NO: 21 - protein, 86 aa, Mus musculus
TLHEFKKSAKTTLTKEDPLLKIKTKKVNSADECANRCIRNRGFTFTCKAFVFDKSRKRCYWYPFNSMSSGVKKGFGHEFDLYENKD

SEQ ID NO: 22 - protein, 87 aa, Homo sapiens
NTIHEFKKSAKTTLIKIDPALKIKTKKVNTADQCANRCTRNKGLPFTCKAFVFDKARKQCLWFPFNSMSSGVKKEFGHEFDLYENKD

SEQ ID NO: 23 - protein, 86 aa, Canis lupus
TLHEFKKSAKTTLTKEDPLLKIKTKKMNTADQCANRCIRNKGLPFTCKAFVFDKARKRCLWFPFNSMSSGVKKEFGHEFDLYENKD

SEQ ID NO: 24 - protein, 86 aa, synthetic construct; identical to SEQ ID NO: 23
  REGION 1..86: note = Feline catus HGF

SEQ ID NO: 25 - protein, 82 aa, Zea mays
DRYDKRSMRFVRLPNTDFWGSDQQHLLSVSLRTCRDICISDCTCKGFQYQEGTGSCYPKAYLFSGRTYPTSDVRTIYLKLPT

SEQ ID NO: 26 - protein, 81 aa, Physcomitrella patens
KNDTFISVTGMTYLGIGLFGCPNSTLAQCKLNCSNDCKCTNALHVKSSLSDSGTCYLMPGEAVQTIRKTGNDTIDLQTLFL

SEQ ID NO: 27 - protein, 81 aa, Arabidopsis thaliana
DGRDGFVRLKKMRLPDTTETSVDKGIGLKECEERCLKGCNCTAFANTDIRNGGSGCVIWSGGLFDIRNYAKGGQDLYVRVA

SEQ ID NO: 28 - protein, 85 aa, Oryza sativa
GNTTTTDGFALVQGVKLPDTHNASVDTGITVEECRARCVANCSCLAYAAADIRGGGGGSGCVIWTGGIVDLRYVDQGQGLFLRLA

SEQ ID NO: 29 - protein, 81 aa, synthetic construct
  REGION 1..81: note = Populus trichocarpa
DKGDRFLQHGGVKLPDMIKSWVDTSKGLKECKDLCLKNCSCVAYANSDIRGGGSGCLLWFDELIDTRELTTGGQDLYIRIA

SEQ ID NO: 30 - protein, 89 aa, Homo sapiens
NTIHEFKKSAKTTLIKIDPALKIKTKKVNTADQCANRCTRNKGLPFTCKAFVFDKARKQCLWFPFNSMSSGVKKEFGHEFDLYENKDYI

SEQ ID NO: 31 - protein, 89 aa, synthetic construct
  REGION 1..89: note = H. sapiens HGF mutated
NTIHEFKKSAKTTLIKIDPALKIKTKKVNTADQAANRATRNKGLPFTAKAFVFDKARKQALWFPFNSMSSGVKKEFGHEFDLYENKDYI

(19) United States
(12) Patent Application Publication
(10) Pub. No.: US 2024/0000956 A1
(43) Pub. Date: Jan. 4, 2024

(54) PAN DOMAIN ACTIVATES UBIQUITINATION

(71) Applicant: UT-Battelle, LLC, Oak Ridge, TN (US)

(72) Inventors: Kuntal De, Oak Ridge, TN (US); Wellington Muchero, Oak Ridge, TN (US); Debjani Pal, Knoxville, TN (US)

(21) Appl. No.: 18/323,672

(22) Filed: May 25, 2023

Related U.S. Application Data
(60) Provisional application No. 63/345,591, filed on May 25, 2022.

(51) Int. Cl. A61K 47/64

(52) U.S. Cl. CPC A61K 47/64

(57) ABSTRACT

The present disclosure is directed to methods for modulating protein function through attaching a PAN domain or a functional fragment thereof to a target protein thereby promoting internalization and degradation of the target protein, wherein the target protein does not comprise a native PAN domain. Another aspect of the current disclosure is directed to a method of inhibiting the internalization of a targeted extracellular protein wherein the targeted extracellular protein comprises a PAN domain.

### CROSS REFERENCE TO RELATED APPLICATION

This application claims the benefit of U.S. Provisional Patent Application No. 63/345,591, filed May 25, 2022, the contents of which are incorporated herein by reference in its entirety.

### STATEMENT REGARDING FEDERALLY SPONSORED RESEARCH OR DEVELOPMENT

This disclosure was made with government support under a research project supported by Prime Contract No. DE-AC05-000R22725 awarded by the U.S. Department of Energy. The government has certain rights in this invention.

### INCORPORATION BY REFERENCE OF SEQUENCE LISTING

The sequence listing in the XML, named as 41946_5064_01_SubstituteSequenceListing.xml of 58 KB, created on Jul. 31, 2023, and submitted to the United States Patent and Trademark Office via Patent Center, is incorporated herein by reference.

### BACKGROUND

Ubiquitin-Proteasome Pathway (UPP) is a regulated machinery that controls the degradation and turnover of 80-90% of proteins in human cells. Its malfunction is linked to many human diseases, from various neurodegenerative dysfunctions and viral infections to cancer. Deregulation of UPP often leads to cancer development. Rapid and timely degradation of the cell cycle regulatory proteins and transcriptional regulators is crucial for the normal functioning of different cellular processes.

Ubiquitin is a protein modifier that plays a central role in all eukaryotic cellular pathways and disease processes, including plant immune responses. It is covalently attached to proteins through an enzymatic cascade involving three enzymes (E 1, E2, E3 enzymes), with the E3s conferring target protein specificity. Nature evolved diverse peptide motifs, termed degrons, to signal substrates for degradation. Each degron provides a specific signal characterized by posttranslational modifications, including phosphorylation, glycosylation, and sumoylation. It is essential to characterize the biochemistry of the E3 ligase interactions with its targets to develop small molecules that can be used to modulate the entire system for therapeutic purposes as well as plant immunity. The ubiquitin-proteasome system (UPS) has also been shown to be an integral part of plant responses to stresses, including plant defense against pathogens. Plant genomes encode many ubiquitin-proteasome (UPS) components compared with other eukaryotes. Arabidopsis genome encodes more than 1600 proteins involved in the ubiquitin-related pathway, underlining the importance of diverse cellular processes. Most of these genes (>1400) encode putative E3-ubiquitin ligases responsible for substrate specificity since they define the substrates for ubiquitination. Intriguingly, we have recently identified the occurrence of the plasminogen-apple-nematode (PAN) domain in more the 28,000 proteins across 2,496 organisms representing 959 genera. A Gene Ontology (GO) enrichment analysis revealed that these proteins were highly enriched in immune responses, including cell recognition, cell communication, proteolysis, pollen pistil recognition, reproduction, response to stimulus, and stress response.

Members of the G-type LecRLK family are increasingly being implicated in enhancement of microbial colonization or parasitic infection in plants. For example, a study showed that constitutive expression of the Populus trichocarpa PtG-type LecRLK1 in Arabidopsis resulted in enhanced colonization by the ectomycorrhizal fungal symbiont Laccaria bicolor, even though Arabidopsis is not a natural host of this fungus (Labbé et al., 2019). In a follow up study, the same PtG-type LecRLK1 was expressed in switchgrass resulting in successful formation of functional mycorrhizae (Qiao et al., 2021). The latter result being especially noteworthy since grasses are not known to form mycorrhizae with ectomycorrhizal fungi in nature. In another study, a G-type LecRLK was reported to function as a susceptibility factor to the fungal pathogen Sphaerulina musiva in P. trichocarpa (Muchero et al., 2018). Besides fungal symbionts and pathogens, a G-type LecRLK was also shown to mediate infection of Arabidopsis by parasitic root-knot nematodes. Loss-of-function mutants of this receptor resulted in enhanced resistance to nematode infection, supporting the role of G-type LecRLKs as negative regulators of immune responses (Zhou et al., 2021). Despite these observed instances of enhanced susceptibility facilitated by G-type LecRLKs, the mechanism by which these receptors function to repress defense signaling and facilitate infection remains entirely unknown. Our study for the first time demonstrates that G-type LecRLKs suppress j asmonic acid (JA) and ethylene (ET) signaling pathways via the PAN domain. A key distinguishing factor between G-type LecRLKs and other defense-associated receptors in plants is the presence of Bulb-lectin (G-lectin), epidermal growth factor (EGF), Plasminogen-Apple-Nematode (PAN) and S-locus protein domains in their extracellular N-terminal region (Acosta and Farmer, 2010). G-type LecRLKs also have the highest copy numbers across plant genomes compared to their C- and L-type LecRLK counterparts.

### SUMMARY

The current disclosure is directed to methods for modulating protein function, by attaching a PAN domain or a functional fragment thereof to a target protein thereby promoting internalization and degradation of the target protein, wherein the target protein does not comprise a native PAN domain. Another aspect of the current disclosure is directed to a method of inhibiting the internalization of a targeted extracellular protein wherein the targeted extracellular protein comprises a PAN domain.

Certain aspects of the current disclosure are directed to a method for modulating protein function, the method comprising modifying a target protein by attaching a PAN domain or a functional fragment thereof to the target protein thereby promoting internalization and degradation of the target protein, wherein the target protein does not comprise a native PAN domain. In some embodiments, the PAN domain comprises SEQ ID NO: 1. In some embodiments, the functional fragment of the PAN domain comprises an amino acid sequence that is at least 90% identical to amino acids 45-80 of SEQ ID NO: 1. In some embodiments, the functional fragment of the PAN domain comprises an amino acid sequence that is at least 90% identical to amino acids 40-85 of SEQ ID NO: 1. In some embodiments, the functional fragment of the PAN domain comprises an amino acid sequence that is at least 90% identical to amino acids 35-90 of SEQ ID NO: 1. In some embodiments, the functional fragment of the PAN domain comprises an amino acid sequence that is at least 90% identical to amino acids 30-95 of SEQ ID NO: 1. In some embodiments, the functional fragment of the PAN domain comprises an amino acid sequence that is at least 90% identical to amino acids 35-80 of SEQ ID NO: 1. In some embodiments, the functional fragment of the PAN domain comprises an amino acid sequence that is at least 90% identical to amino acids 45-90 of SEQ ID NO: 1. In some embodiments, the target protein is involved in the RAF/MEK/ERK signaling cascade. In some embodiments, the target protein is selected from C-RAF, ERK, and MAPK. In some embodiments, the target protein is involved in the IGF/Akt signaling pathway. In some embodiments, the target protein is selected from Akt, mTOR, p53, and PARP. In some embodiments, the target protein is Kif11 protein. In some embodiments, the target protein is associated with a pathogen. In some embodiments, the target protein is SylA. In some embodiments, the target protein is SKP1.

In some embodiments, the method comprises modifying a target protein by attaching a PAN domain or a functional fragment thereof to the target protein thereby promoting internalization and degradation of the target protein, wherein the target protein does not comprise a native PAN domain, and wherein the PAN domain comprises an amino acid sequence that is at least 90% identical to SEQ ID NO: 2. In some embodiments, the functional fragment of the PAN domain comprises an amino acid sequence that is at least 90% identical to amino acids 70-96 of SEQ ID NO: 2. In some embodiments, the functional fragment of the PAN domain comprises an amino acid sequence that is at least 90% identical to amino acids 65-101 of SEQ ID NO: 2. In some embodiments, the functional fragment of the PAN domain comprises an amino acid sequence that is at least 90% identical to amino acids 60-106 of SEQ ID NO: 2. In some embodiments, the functional fragment of the PAN domain comprises an amino acid sequence that is at least 90% identical to amino acids 55-111 of SEQ ID NO: 2. In some embodiments, the functional fragment of the PAN domain comprises an amino acid sequence that is at least 90% identical to amino acids 60-96 of SEQ ID NO: 2. In some embodiments, the functional fragment of the PAN domain comprises an amino acid sequence that is at least 90% identical to amino acids 70-106 of SEQ ID NO: 2. In some embodiments, the target protein is involved in the RAF/MEK/ERK signaling cascade. In some embodiments, the target protein is selected from C-RAF, ERK, and MAPK. In some embodiments, the target protein is involved in the IGF/Akt signaling pathway. In some embodiments, the target protein is selected from Akt, mTOR, p53, and PARP. In some embodiments, the target protein is Kif11 protein. In some embodiments, the target protein is associated with a pathogen. In some embodiments, the target protein is SylA. In some embodiments, the target protein is SKP1.

Some aspects of the current disclosure are directed to methods for promoting the internalization of a target extracellular protein without a PAN domain, the method comprising: attaching a PAN domain or a functional fragment thereof to a ligand that binds the target extracellular protein, and bringing the ligand attached with the PAN domain into contact with the target extracellular protein to permit binding of the ligand to the target extracellular protein, thereby initiating the internalization of the target extracellular protein. In some embodiments, the PAN domain comprises SEQ ID NO: 1. In some embodiments, the functional fragment of the PAN domain comprises an amino acid sequence that is at least 90% identical to amino acids of SEQ ID NO: 1. In some embodiments, the PAN domain comprises SEQ ID NO: 2. In some embodiments, the functional fragment of the PAN domain comprises an amino acid sequence that is at least 90% identical to amino acids 70-96 of SEQ ID NO 2. In some embodiments, the target extracellular protein is selected from microbe-associated molecular patterns (MAMPs). In some embodiments, the active PAN domain is attached to a PRR, thereby initiating ubiquitination of the PRR. In some embodiments, the MAMPs are from bacteria or fungi. In some embodiments, the fungi are selected from Paecilomyces tenuipes and Beauveria bassiana.

Another aspect of the disclosure is directed to a method for inhibiting the internalization of a targeted extracellular protein wherein the targeted extracellular protein comprises a PAN domain, the method comprising inactivating the PAN domain of the targeted extracellular protein. In some embodiments, inactivating the PAN domain of the targeted extracellular protein is completed by substituting at least one of the four conserved cysteines of SEQ ID NO: 1. In some embodiments, inactivating the PAN domain of the targeted extracellular protein is completed by substituting at least two of the four conserved cysteines of SEQ ID NO: 1. In some embodiments, inactivating the PAN domain of the targeted extracellular protein is completed by substituting at least three of the four conserved cysteines of SEQ ID NO: 1. In some embodiments, inactivating the PAN domain of the targeted extracellular protein is completed by substituting at each of the four conserved cysteines of SEQ ID NO: 1. In some embodiments, inactivating the PAN domain of the targeted extracellular protein is completed by substituting at least one of the four conserved cysteines of SEQ ID NO: 2. In some embodiments, inactivating the PAN domain of the targeted extracellular protein is completed by substituting at least two of the four conserved cysteines of SEQ ID NO: 2. In some embodiments, inactivating the PAN domain of the targeted extracellular protein is completed by substituting at least three of the four conserved cysteines of SEQ ID NO: 2. In some embodiments, inactivating the PAN domain of the targeted extracellular protein is completed by substituting each of the four conserved cysteines of SEQ ID NO: 2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B. The HGF PAN domain is comprised of a core of four conserved cysteine residues. A. Multiple alignment of the sequences of PAN domain of representative proteins from different organisms highlights the position of four conserved cysteines. B. Schematic diagram of amino acid sequence represents HGF PAN domain along with the four marked conserved cysteines (Cys70, Cys74, Cys84 and Cys96) and the subsequent mutant version where conserved cysteines were mutated to alanine (Ala70, Ala74, Ala84 and Ala96). Computational secondary structure prediction (Chimera) for HGF PAN domain identifies the residues forming β-strands and α-helix in PAN domain.

FIG. 2A-G. PAN domain modulates HGF stability. A, B. Core cysteines in PAN domain are crucial for HGF stability. A. Immunoblot analysis of whole cell lysates derived from 293T cells, transfected with Flag-HGF WT and different single cysteine mutants of Flag-HGF constructs as indicated. 30 h post-transfection, whole-cell lysates were prepared for immunoblot analysis. Representative image of n=3 biological replicates. B. Quantification of the band intensities in (A). The intensities of Flag-HGF (WT and mutants) bands were normalized to actin and then normalized to Flag-HGF WT. Data are represented as mean±SEM, n=3, and *p<0.05, **p<0.005, ***p<0.0005 (student's t-test). C. Mutation in all four core cysteine residues in PAN domain remarkably alters HGF stability. Immunoblot analysis of whole cell lysates derived from 293 T cells, transfected with Flag-HGF WT and Flag-HGF 4Cys-4Ala constructs as indicated. 30 h post-transfection, whole-cell lysates were prepared for immunoblot analysis. D. Cysteines in kringle domains and SPH domain of HGF have no impact on the protein abundance in cells. Immunoblot analysis of whole cell lysates derived from 293T cells, transfected with Flag-HGF WT, Flag-HGF 4Cys-4Ala, and different single cysteine mutants of Flag-HGF constructs as indicated. 30 h post-transfection, whole-cell lysates were prepared for immunoblot analysis. Representative image of n=3 biological replicates. E. Quantification of the band intensities in (D). The intensities of Flag-HGF (WT and mutants) bands were normalized to actin and then normalized to Flag-HGF WT. Data are represented as mean±SEM, n=3, and *p<0.05, **p<0.005, ***p<0.0005 (student's t-test). F. Localization of Flag-HGF WT and different Flag-HGF mutants' expression by confocal immunofluorescence microscopy in HeLa cells. The cells were transiently transfected with Flag-HGF WT and different mutants of Flag-HGFs as indicated. 30 h post-transfection cells were fixed, mounted and protein expression patterns were visualized using a Zeiss LSM 710 confocal microscope outfitted with a 63× objective. Scale bars represent 20 μm. The images shown are representative from three independent biological experiments (average 100 cells were observed per experimental condition per replicate). G. Percentage of transfected HeLa cells showing perinuclear/nuclear staining for Flag-HGF WT and Flag-HGF 4Cys-4Ala were quantified. Data are represented as mean±SD, n=3 (average 100 cells were observed for each condition per experiment), and *p<0.05, **p<0.005, ***p<0.0005 (Student's t test).

FIG. 3A-E: HGF PAN domain regulates c-MET signaling cascade via four core cysteines. A. Mutation of the core cysteines in HGF PAN domain blocks HGF-induced c-MET signaling. Both 293T and U-87 MG cells were stimulated with HGF WT and HGF 4Cys-4Ala proteins for the indicated amount of time. Cells were harvested and immunoblot analysis shows the absence of phosphorylation for c-MET, AKT, and ERK in presence of HGF 4Cys-4Ala. Representative blot images from n=2 experiments for individual cell line. B. Immunoblot showing that the core cysteines on the HGF PAN domain regulate its binding with c-MET. c-MET was immunoprecipitated from 293T cells on anti-MET bound beads. In-vitro translated Flag-tagged HGF WT and HGF 4Cys-4Ala were added to the beads as indicated to detect the interaction between endogenous c-MET and Flag-HGF WT and Flag-HGF 4Cys-4Ala. C. Right panel, quantification of the band intensities (n=2; ***p<0.0005 (Student's t test)). Immunoprecipitated Flag-HGFs band intensities were normalized to the respective c-MET IP bands and then further normalized to HGF-WT. D. HGF PAN domain defines perinuclear translocation of c-MET in cells. HeLa cells were stimulated with HGF WT and HGF 4Cys-4Ala proteins for the indicated amount of time following serum starvation. Post stimulation cells were fixed, mounted and endogenous c-MET localization pattern was visualized using Zeiss LSM 710 at 63× objective. Scale bars represent 20 μm. The images shown are representative from three independent biological replicates (average 100 cells were observed for each condition per replicate). E. PAN domain regulates c-MET ubiquitination. In vivo ubiquitination assay shows that HGF WT promotes c-MET ubiquitination in a PAN-dependent manner. 293T cells were transfected with the construct c-MET-C-GFPSpark. After serum starvation, cells were stimulated with HGF WT and HGF 4Cys-4Ala as indicated. The lysates were collected at specific time points and incubated with anti-GFP protein G beads. Ubiquitinated-c-MET proteins were eluted, resolved by SDS-PAGE, and immunoblotted with the indicated antibodies.

FIG. 4A-B. Core cysteines in HGF PAN domain is essential for STAT3 phosphorylation and nuclear translocation. A. Impaired HGF PAN domain is unable to initiate STAT3 phosphorylation. U-87 MG cells were treated with HGF WT and HGF 4Cys-4Ala where indicated for 1, 2, and 4 h. Cell extracts were prepared and probed for phosphor-STAT3 and total STAT3. B. STAT3 nuclear localization is suppressed by PAN mutant HGF. HeLa cells were stimulated with HGF WT and HGF 4Cys-4Ala as indicated. Cells were fixed and STAT3 was immunostained with a STAT3-specific antibody. The localization of STAT3 (green) and 4,6-diamidino-2-phenylindole (DAPI) (blue) in U-87 MG cells. Images were visualized using Zeiss LSM 710 at 63× objective. Scale bars represent 20 μm. The images shown are representative of three independent biological replicates (an average of 100 cells were observed for each condition per replicate).

FIG. 5A-C: Transcriptome analysis post HGF stimulation in 293T cells. Differential expression analysis by RNA seq in 293T cells following HGF WT and HGF 4Cys-4Ala treatment confirms that core cysteines in HGF PAN domain are necessary for the expression of a wide range of genes. Responsive genes were normalized to FPKM value for non-treated cells and then normalized to HGF WT treated cells. Data represents the average of three independent biological replicates and *p<0.05, **p<0.005 and ***p<0.0005 were calculated with a student's t-test. A. Shows differential expression analysis for MET signaling pathway proteins. B. Shows differential expression analysis for cell cycle proteins. C. Shows differential expression analysis for known pathways in cancer.

FIG. 6A-C: GLecRLK PAN domain is comprised of conserved amino acid residues and is critical for immunosuppression in plants. A. Multiple sequence alignment within the GLecRK PAN domains and naturally occurring variation in non-immunosuppressive Salix orthologs SapurV1A.0918s0020 (SpG-type LecRLK-1) and SapurV1A.0037s0270.1 (SpG-type LecRLK-2). B. Multiple sequence alignment with the G-LecRK PAN domains and naturally occurring variation in nonimmunosuppressive Salix orthologs SapurV1A.0918s0020 (SpG-type LecRLK-1) and SapurV1A.0037s0270.1 (SpG-type LecRLK-2) with their respective restored version. C. Schematic representation of typical G-type LecRLKs, G-type LecRLKs with PAN domain mutations (SpG-type LecRLK-1 & SpG-type LecRLK-2) and G-type LecRLKs with restored PAN domains (SpGtype LecRLK-1R & SpG-type LecRLK-2R).

FIG. 7A-F: G-type RLKs suppresses WRKY33 transcription. A and B. Transcriptome data from Arabidopsis transgenic plants shows differential expression of WRKY33 in transgenic Arabidopsis plants. Results are shown as mean±SE of four different independent experiments. Asterisks represents significant differences between transgenic plants and empty vector control (***P<0.0005; **P<0.005; *P<0.05 Student T-test). C. Transcriptome data from Nicotiana transgenic plants shows differential expression of WRKY33. Results are shown as mean±SE of three different independent experiments. Asterisks represents significant differences between transgenic plants and wild type control (***P<0.0005; **P<0.005; *P<0.05 Student T-test). D and E. Transcriptome data from Arabidopsis transgenic plants shows differential expression of MPK3. Results are shown as mean±SE of four different independent experiments. Asterisks represents significant differences between transgenic plants and empty vector control (***P<0.0005;**P<0.005; *P<0.05 Student T-test). F. Transcriptome data from Nicotiana transgenic plants shows MPK3 is significantly upregulated in SpGtype LecRLK-1. MAPK is hyperphosphorylated in transgenic SpG-type LecRLK-1 compared to SpG-type LecRLK-1R Nicotiana plants. Representative image of 2 independent experiments which shows similar results.

FIG. 8A-D: PAN domain specifically suppresses JA and ET pathway. A. Differential expression of genes involved in JA pathway and in ET pathway in Arabidopsis. Results are shown as mean±SE (n=4 biological replicates). Asterisks represents significant differences between transgenic plants and empty vector control (***P<0.0005; **P<0.005; *P<0.05 Student T-test). B. Differential expression of genes involved in JA pathway and in ET pathway in Tobacco. Results are shown as mean±SE (n=3 biological replicates). C. Reduced expression of negative regulators in mutated PAN transgenic version of SpG-type LecRLK-1 and SpG-type LecRLK-2. Results are shown as mean±SE (n=4 biological replicates). D. Mode of action showing mutating PAN domain repress TOPLESS, HDA6 and NINJA which ultimately triggers JA pathway.

FIG. 9A-D: Functional PAN domain regulates the phytohormone level in plants. A. and B. Hormone analysis determines jasmonic acid and jasmonoyl-isoleucine (JA-IIe) hormone levels were significantly higher in SpG-type LecRLK-1 and SpG-type LecRLK-2 transgenic Arabidopsis while the restored variants had control levels. C and D. Salicylic acid and abscisic acid exhibited negligible changes in four variants. Results are shown as mean±SE (n=3 biological replicates). Asterisks represents significant differences between transgenic plants and empty vector control (***P<0.0005; **P<0.005; *P<0.05 Student T-test).

FIG. 10A-H: PAN domain modulates ROS response and immune suppression to Botrytis. A and B. Expression level of two marker genes which includes Botrytis induced kinase 1 (BIK1) and RBOHD. Bars represent the standard error of the mean for four biological replicates. Statistical difference was determined by two-tailed student's T test against Col-0 (***P<0.0005; **P<0.005; *P<0.05 Student Ttest). C and D. Flg22 elicitation assay: Oxidative burst triggered by 500 nM flg22 in wild type Col-0, SpG-type LecRLK-1 and SpG-type LecRLK-1R transgenic plants measured in relative light units (RLU). Data represents standard deviation of the mean from three independent experiments from 9 biological replicates. D. Oxidative burst triggered by 500 nM flg22 in wild type Col-0, SpGtype LecRLK-2 and SpG-type LecRLK-2R transgenic plants measured in relative light units (RLU). Data represents standard deviation of the mean from three independent experiments from 9 biological replicates. E. Botrytis infection assay: Botrytis cinerea inoculation of Col-0, SpG-type LecRLK-1 and SpGtype LecRLK-1R transgenic Arabidopsis plants. F. Lesion area (percentage of total leaf area) in 72 hours post inoculation and 96 hours post inoculation are represented in bar graphs. Value represents mean±SE from at least n=20 from each group. Asterisks represents significant differences (***P<0.0005; **P<0.005; *P<0.05 Student T-test). G. Reactive oxygen species detection by confocal laser scanning microscopy in Arabidopsis leaves. Merged two-color confocal images of the green and the red channel show ROS detected by carboxy-H2DCFDA probe (green) and autofluorescence of chloroplast (red). The experiment is the representative image of 3 biological replicates. Size Bar: 10 um. H. Working model showing mutated PAN triggers immunity (left) and restored version dampens immunity (right).

FIG. 11A-D: PAN domain is critical for G-type LECRLK phosphorylation and ubiquitination. A. GFP-tagged SpG-type LecRLK-1 and GFP-tagged SpG-type LecRLK-1R protein were detected with GFP antibody and probed subsequently with phosphoserine antibody. 3 biological replicates are shown. B. Band intensity were quantified using a densitometer and plotted relative intensities. Data are shown as mean±SEM of three different independent experiments. Asterisks represents significant differences between transgenic mutant PAN containing G-type LECRLK and restored PAN containing G-type LecRLK plants (*P<0.05 Student T-test). C. Heat map of the differential expression analysis from RNA seq shows expression of E3 ubiquitin ligases in the empty vector, SpG-type LecRLK-1, SpG-type LecRLK-1R, SpG-type LecRLK-2 and SpG-type LecRLK-2R. D. 35S HA SpG-type LecRLK-land 35S HA SpG-type LecRLK-1R transformed protoplast were treated with cycloheximide together with MG132 for 0, 2 and 4 hours. Ubiquitinated and total SpGtype LecRLK-1 and SpG-type LecRLK-1R proteins were detected with HA antibody on the lysate. The membrane was re-probed with ubiquitin specific antibody. Data are shown as representative of two different independent experiments.

FIG. 12A-D: Conserved cysteine residues in PAN domain are critical for immunosuppression in plants. A. Schematic representation of transgenic SpG-type LecRLK-1R6cys-6ala construct from SpG-type LecRLK-1R. B. Quatitative real time PCR data from Arabidopsis transgenic plants shows induced expression of WRKY33 in transgenic SpG-type LecRLK-1R6cys-6ala Arabidopsis plants. Results are shown as mean±SE of three different independent experiments. Asterisks represents significant differences between transgenic plants and wild type control (*P<0.05 Student T-test). C. Schematic representation of transgenic SpG-type LecRLK-2R6cys-6ala construct from SpG-type LecRLK-2R. D. Quatitative real time PCR data from Arabidopsis transgenic plants shows induced expression of WRKY33 in transgenic SpG-type LecRLK-2R6cys-6ala Arabidopsis plants. Results are shown as mean±SE of three different independent experiments. Asterisks represents significant differences between transgenic plants and wild type control (*P<0.05 Student T-test).

FIG. 13A-C: Working model to show the function of G-type LecRLK containing PAN domains in plants. A. Schematic diagram indicates active PAN domain in transgenic SpG-type LecRLK-1 and SpG-type LecRLK-2 unable to activate JA and ET signaling. B. mutation in conserved amino acid residues in PAN domain able to trigger the pathway. C. Working model showing the functional PAN domain of G-LecRLKs can self-interact and form higher order oligomers which gets targeted by ubiquitination and further suppresses plant immunity in host. On the other side, mutation in the conserved PAN domain of G-LecRLKs unable to form higher order oligomers and triggers host immunity in plants.

### DETAILED DESCRIPTION

Although claimed subject matter will be described in terms of certain examples, other examples, including examples that do not provide all the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

“Complementary” or “substantially complementary” refers to the hybridization or base pairing or the formation of a duplex between nucleotides or nucleic acids, such as, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the other strand, usually at least about 90% to about 95% (e.g., at least 90%, at least 91%, at least 92%, at least 93%, at least 94%, and at least 95%), and even at least about 98% to about 100% (e.g., at least 98%, at least 99%, and 100%).

“Hybridization” refers to the process in which two single-stranded polynucleotides bind non-covalently to form a stable double-stranded polynucleotide. The resulting (usually) double-stranded polynucleotide is a “hybrid” or “duplex.” “Hybridization conditions” will typically include salt concentrations of approximately up to 1M, often up to about 500 mM and may be up to about 200 mM. A “hybridization buffer” is a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a primer will hybridize to its target subsequence but will not hybridize to the other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tm for the specific sequence at a defined ionic strength and pH. Exemplary stringent conditions include a salt concentration of at least 0.01 M to no more than 1M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately C. are suitable for allele-specific hybridizations, though a suitable temperature depends on the length and/or GC content of the region hybridized.

“Nucleic acid”, “oligonucleotide”, “oligo” or grammatical equivalents used herein refers generally to at least two nucleotides covalently linked together. A nucleic acid generally will contain phosphodiester bonds, although in some cases nucleic acid analogs may be included that have alternative backbones such as phosphoramidite, phosphorodithioate, or methylphophoroamidite linkages; or peptide nucleic acid backbones and linkages. Other analog nucleic acids include those with bicyclic structures including locked nucleic acids, positive backbones, non-ionic backbones and non-ribose backbones. Modifications of the ribose-phosphate backbone may be done to increase the stability of the molecules; for example, DNA: DNA hybrids can exhibit higher stability in some environments.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

As used herein, the phrase “targeted protein” or “target protein” refers to a protein that regulates a cell's response to a foreign entity.

The term “homolog” or “homologous” means a gene related to a second gene by descent from a common ancestral DNA sequence, therefore, the corresponding polynucleotide/polypeptide has a certain degree of homology, i.e., sequence identity (at least 40%, 60%, 65%, at least 66%, 68%, 70%, 75%, 80%, 86%, 88%, 90%, 92%, 95%, 97% or 99% sequence identity). A “homolog” furthermore means that the function is equivalent to the function of the original gene. Homologs of a given gene and corresponding or equivalent positions in the homologous genes or proteins can be determined by sequence alignment programs, e.g., including but not limited to, NCBI BLAST, ClustalW, DIAMOND, CS-BLAST, and MAFFT.

As used herein, “nonconservative mutation” refers to a change in a DNA or RNA sequence that results in the replacement of an amino acid with one that is not biochemically similar. In some embodiments, a nonconservative mutation of a cysteine residue comprises changing the cysteine residue to any amino acid other than serine, selenocysteine, threonine, and methionine (because serine, selenocysteine, threonine, and methionine are biochemically similar to cysteine). In some embodiments, the non-silent mutation is a nonconservative mutation. In some embodiments, the nonconservative mutation is a cysteine to alanine mutation.

Targeted Genome Engineering

Targeted genome engineering (also known as genome editing) has emerged as an alternative to classical plant breeding and transgenic (Genetically Modified Organism—GMO) methods to improve crop plants. Available methods for introducing site-specific double strand DNA breaks include zinc finger nucleases (ZFNs), TAL effector nucleases (TALENs) and CRISPR/Cas system. ZFNs are reviewed in Carroll, D. (Genetics, 188.4 (2011): 773-782), and TALENs are reviewed in Zhang et al. (Plant Physiology, 161.1 (2013): 20-27), which are incorporated herein in their entirety.

CRISPR/Cas system is a method based on the bacterial type II CRISPR (clustered regularly interspaced short palindromic repeats)/Cas (CRISPR-associated) immune system. The CRISPR/Cas system allows targeted cleavage of genomic DNA guided by a customizable small noncoding RNA, resulting in gene modifications by both non-homologous end joining (NHEJ) and homology-directed repair (HDR) mechanisms. Belhaj et al. (Plant Methods, 2013, 9:39) summarizes and discusses applications of the CRISPR/Cas technology in plants and is incorporated herein in its entirety.

In some embodiments, genome editing is achieved by CRISPR (Clustered regularly-interspaced short palindromic repeats)/Cas technology. CRISPR-Cas and similar gene targeting systems are well known in the art with reagents and protocols readily available. Exemplary genome editing protocols are described in Jennifer Doudna, and Prashant Mali, “CRISPR-Cas: A Laboratory Manual” (2016) (CSHL Press, ISBN: 978-1-621821-30-4) and Ran, F. Ann, et al. (Nature Protocols (2013), 8 (11): 2281-2308).

The Plasminogen Apple Nematode (PAN) Domain

The Plasminogen-Apple-Nematode (PAN) domain (Pfam ID: PF00024; InterPro ID: IPR003609; PROSITE ID: PD0000376) was first characterized by Tordai et al., (FEBS Letters, 461(1-2), 63-67, (1999)) in which they noted that the domain was shared by the plasminogen/hepatocyte growth factor protein family, the prekallikrein/coagulation factor XI protein family and nematode proteins. The PAN domain contains 4 to 6 conserved cysteine residues. The conserved residues are involved in the formation of disulfide bond links between the first and sixth, second and fifth, third and fourth cysteines to form a hairpin-loop structure. Cysteine residues 1 and 6 are not conserved in some PAN domain containing proteins such as the Hepatocyte Growth Factor (HGF). As used herein, a PAN domain has the following consensus sequence: C-x(3)-[LIVMFY]-x(5)-[LIVMFY]-x(3)-[DENQ]-[LIVMFY]-x(10)-C-x(3)-C-T-x(4)-C-x-[LIVMFY]-F-x-[FY]-x(13-14)-C-x-[LIVMFY]-[RK]-x-[S1]-x(14-15)-S-G-x-[ST]-[LIVMFY]-x(2)-C(SEQ ID NO: 1). In some embodiments, the consensus sequence has the sequence as shown in any one of SEQ ID NO: 3-5. The skilled artisan will be able to determine whether a protein comprises a PAN domain by searching the amino acid sequence of the protein using the consensus PAN domain sequence as a query sequence and any sequence alignment software.

Over 28,000 proteins across eukaryotes, archaea, bacteria and viruses carrying the PAN domain have been curated for this disclosure. See List 1 from U.S. Pat. Publication No. 2021/0072228A1. After classifying 2,496 organisms representing 959 genera, into 13 categories (Alveolata, Archea, Amoebazoa, Bacteria, Cryptophyta, Euglenozoa, Haptophyceae, Opisthokonta, Rhizaria, Rhodophyta, Stramenopilles, Viridplantae, and Viruses), it was observed that proteins carrying the PAN domain have been implicated in modulation of immune responses across divergent organisms (e.g., from plants to animals, from viruses to bacteria and fungi), suggesting that the domain may have been co-opted to serve the same immunosuppressive function under different biological circumstances.

Modulation of Protein Function

Certain aspects of the current disclosure are directed to methods for modulating protein function. Protein function can be modulated through the PAN domain. One aspect of the current disclosure is a method for modulating protein function by attaching a PAN domain or a functional fragment of a PAN domain to a target protein that does not comprise a native PAN domain. The attachment of a PAN domain or functional fragment thereof promotes the internalization and degradation of the target protein.

In some embodiments, the PAN domain comprises the consensus sequence of SEQ ID NO: 1: C-x(3)-[LIVMFY]-x(5)-[LIVMFY]-x(3)-[DENQ]-[LIVMFY]-x(10)-C-x(3)-C-T-x(4)-C-x-[LIVMFY]-F-x-[FY]-x(13-14)-C-x-[LIVMFY]-[RK]-x-[ST]-x(14-15)-S-G-x-[ST]-[LIVMFY]-x(2)-C.

In some embodiments, the PAN domain comprises an amino acid sequence that is at least 80%, 85%, 90%, 91%, 92%, 93%, 94%, 95%, 96%, 97%, 98%, 99% or 100% identical to the amino acid sequence of SEQ ID NO: 1, or a functional fragment thereof. As used herein, “functional fragment” is a term of art that refers to a portion of the full amino acid sequence that has the same function as the full amino acid sequence. In some embodiments, the functional fragment of the PAN domain comprises amino acids 45-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-80 of SEQ ID NO: 1. In some embodiments, the functional fragment of the PAN domain comprises amino acids 40-85 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 1. In some embodiments, the functional fragment of the PAN domain comprises amino acids 35-90 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 35-90 of SEQ ID NO: 1. In some embodiments, the functional fragment of the PAN domain comprises amino acids 30-95 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 30-95 of SEQ ID NO: 1. In some embodiments, the functional fragment of the PAN domain comprises amino acids 35-80 of SEQ ID NO: 1. In some embodiments, the functional fragment of the PAN domain comprises amino acids 45-90 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-90 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-95 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-100 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-105 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-110 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-115 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-120 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 40-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 35-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 25-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 20-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 15-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 10-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 1-80 of SEQ ID NO: 1.

In some embodiments, the PAN domain comprises the amino acid sequence of SEQ ID NO: 2:

MWVTKLLPALLLQHVLLHLLLLPIAIPYAEGQRKRRNTIHEFKKSAKTTL
IKIDPALKIKTKKVNTADQCANRCTRNKGLPFTCKAFVFDKARKQCLWFP
FNSMSSGVKKEFGHEFDLYENKDYI.

In some embodiments, the PAN domain comprises an amino acid sequence that is at least 80%, 85%, 90%, 91%, 92%, 93%, 94%, 95%, 96%, 97%, 98%, 99% or 100% identical to the amino acid sequence of SEQ ID NO: 2, or a functional fragment thereof. In some embodiments, the functional fragment of the PAN domain comprises amino acids 70-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-96 of SEQ ID NO: 2. In some embodiments, the functional fragment of the PAN domain comprises amino acids 65-101 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 65-101 of SEQ ID NO: 2. In some embodiments, the functional fragment of the PAN domain comprises amino acids 60-106 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 60-106 of SEQ ID NO: 2. In some embodiments, the functional fragment of the PAN domain comprises amino acids 55-111 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 55-111 of SEQ ID NO: 2. In some embodiments, the functional fragment of the PAN domain comprises amino acids 60-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 60-96 of SEQ ID NO: 2. In some embodiments, the functional fragment of the PAN domain comprises amino acids 70-106 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 65-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 60-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 55-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 50-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 40-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 35-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 30-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 25-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 15-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 10-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 5-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 1-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-101 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-106 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-111 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-116 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-121 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-125 of SEQ ID NO: 2.

The PAN domain may be attached to the target protein by ways known in the art, i.e. crosslinking. In some embodiments, the PAN domain is connected to the target protein through a linker. In some embodiments, the linker is a chemical linker. In some embodiments, the linker is a flexible alkyl linker. In some embodiments the linker is chosen from polyethylene glycol (PEG), alkyl, glycol, alkyne, tiazole, piperazine, piperidine or a combination of those. In some embodiments, the linker is an alkyl and/or ether chain comprising between 1 and 30 atoms in length. In some embodiments, the linker is an alkyl and/or ether chain between 3 and 29 atoms in length. In some embodiments, the linker is an alkyl and/or ether chain between 7 and 29 atoms in length. In some embodiments, the linker is an alkyl and/or ether between 3 and 19 atoms in length. In some embodiments, the linker is an alkyl halide. In some embodiments, the linker is a hydrocarbon linker. In some embodiments, the linker is an all-hydrocarbon linker. In some embodiments, the linker is a rigid, polar linker.

In some embodiments, the linker is able to undergo click chemistry. A non-limiting example of a linker that can undergo click chemistry is a tiazole moiety. In some embodiments, the linker comprises an amine linked by amide bond formation or SNAr and an azide with the ability for coupling to alkyne moieties through click chemistry. In some embodiments, the linker is an alkyne and tetrazine precursor known in the art as a CLIPTAC.

In some embodiments, the linker is a photoswitchable linker. A photoswitchable linker can be controlled in a spatiotemporal manner through light stimuli. In some embodiments, azobenzenes, which can reversibly undergo cis-trans isomerisation upon irradiation at different wavelengths are used as the photoswitch.

In some embodiments, the PAN domain will bind to an E3 ubiquitin ligase and a ligand thereby binding to a protein of interest through a chemical linker. In such embodiments the protein of interest is then degraded. In some embodiments, synthetically tractable alkyl and polyethylene glycol as a linker.

In some embodiments, the linker must be optimized based on structural biology and pharmacokinetic properties, notably cell permeability, metabolic stability, and solubility. In some embodiments, linker optimization comprises determining the affinity of the PAN-linker-ligand to recruit the target protein.

In some embodiments, the target protein is involved in the RAF/MEK/ERK signaling cascade. In some embodiments, the target protein is selected from C-RAF, ERK, and MAPK. In some embodiments, the target protein is involved in the IGF/Akt signaling pathway. In some embodiments, the target protein is selected from Akt, mTOR, p53, and PARP. In some embodiments, the target protein is Kif11 protein. In some embodiments, the target protein is associated with a pathogen. In some embodiments, the target protein is SylA. In some embodiments, the target protein is SKP1.

In some embodiments, the method comprises modifying a target protein by attaching a PAN domain or a functional fragment thereof to the target protein thereby promoting internalization and degradation of the target protein, wherein the target protein does not comprise a native PAN domain, and wherein the PAN domain comprises SEQ ID NO: 2. In some embodiments, the functional fragment of the PAN domain comprises amino acids 70-96 of SEQ ID NO: 2. In some embodiments, the target protein is involved in the RAF/MEK/ERK signaling cascade. In some embodiments, the target protein is selected from C-RAF, ERK, and MAPK. In some embodiments, the target protein is involved in the IGF/Akt signaling pathway. In some embodiments, the target protein is selected from Akt, mTOR, p53, and PARP. In some embodiments, the target protein is kinesin family member 11 (Kif11) protein. In some embodiments, the target protein is associated with a pathogen. In some embodiments, the target protein is SKP1.

In some embodiments, the target protein is syringolinA (SylA). Pseudomonas syringae (Pss) secretes SylA, which hinders eukaryotic proteasomes and manipulates the host ubiquitin system. Attaching a PAN domain to the SylA would help in degradation by the host ubiquitin system, enabling the plants to fight pathogen attack. Pathogenic attack can come from bacteria or viruses.

Some aspects of the current disclosure are directed to a method for promoting the internalization of a target extracellular protein without a PAN domain, the method comprising: attaching a PAN domain or a functional fragment thereof to a ligand that binds the target extracellular protein, and bringing the ligand attached with the PAN domain into contact with the target extracellular protein to permit binding of the ligand to the target extracellular protein, thereby initiating the internalization of the target extracellular protein by a cell.

In some embodiments, the PAN domain comprises SEQ ID NO: 1. In some embodiments, the functional fragment of the PAN domain comprises amino acids 45-80 of SEQ ID NO: 1. In some embodiments, the PAN domain comprises SEQ ID NO: 2. In some embodiments, the functional fragment of the PAN domain comprises amino acids 70-96 of SEQ ID NO 2.

In some embodiments, the PAN domain comprises an amino acid sequence that is at least 80%, 85%, 90%, 91%, 92%, 93%, 94%, 95%, 96%, 97%, 98%, 99% or 100% identical to the amino acid sequence of SEQ ID NO: 1, or a functional fragment thereof. As used herein, “functional fragment” is a term of art that refers to a portion of the full amino acid sequence that has the same function as the full amino acid sequence. In some embodiments, the functional fragment of the PAN domain comprises amino acids 45-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-80 of SEQ ID NO: 1. In some embodiments, the functional fragment of the PAN domain comprises amino acids 40-85 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 1. In some embodiments, the functional fragment of the PAN domain comprises amino acids 35-90 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 35-90 of SEQ ID NO: 1. In some embodiments, the functional fragment of the PAN domain comprises amino acids 30-95 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 30-95 of SEQ ID NO: 1. In some embodiments, the functional fragment of the PAN domain comprises amino acids 35-80 of SEQ ID NO: 1. In some embodiments, the functional fragment of the PAN domain comprises amino acids 45-90 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-90 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-95 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-100 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-105 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-110 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-115 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 45-120 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 40-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 35-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 25-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 20-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 15-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 10-80 of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 1. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 1-80 of SEQ ID NO: 1.

In some embodiments, the PAN domain comprises the amino acid sequence of SEQ ID NO: 2. In some embodiments, the PAN domain comprises an amino acid sequence that is at least 80%, 85%, 90%, 91%, 92%, 93%, 94%, 95%, 96%, 97%, 98%, 99% or 100% identical to the amino acid sequence of SEQ ID NO: 2, or a functional fragment thereof. In some embodiments, the functional fragment of the PAN domain comprises amino acids 70-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-96 of SEQ ID NO: 2. In some embodiments, the functional fragment of the PAN domain comprises amino acids 65-101 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 65-101 of SEQ ID NO: 2. In some embodiments, the functional fragment of the PAN domain comprises amino acids 60-106 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 60-106 of SEQ ID NO: 2. In some embodiments, the functional fragment of the PAN domain comprises amino acids 55-111 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 55-111 of SEQ ID NO: 2. In some embodiments, the functional fragment of the PAN domain comprises amino acids 60-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 60-96 of SEQ ID NO: 2. In some embodiments, the functional fragment of the PAN domain comprises amino acids 70-106 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 65-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 60-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 55-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 50-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 40-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 35-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 30-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 25-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 15-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 10-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 5-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 1-96 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-101 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-106 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-111 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-116 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-121 of SEQ ID NO: 2. In some embodiments, the functional fragment of the PAN domain comprises an amino acid sequence that is at least 80%, 85%, 90%, 91%, 92%, 93%, 94%, 95%, 96%, 97%, 98%, 99% or 100% identical to amino acids 70-125 of SEQ ID NO: 2.

In some embodiments, the target extracellular protein is selected from microbe-associated molecular patterns (MAMPs). In some embodiments, the active PAN domain is attached to a plasma membrane bound pattern recognition receptor (PRR), thereby initiating ubiquitination of the PRR. Immunoregulation has been well recorded in plants and PRR are integral parts of surveillance system against pathogens. In some embodiments, the ubiquitination of PRR leads to internalization of beneficial microbes and fungi.

In some embodiments, the MAMPs are from bacteria or fungi. In some embodiments, the fungi are selected from Paecilomyces tenuipes and Beauveria bassiana.

Inhibition of Protein Internalization

Another aspect of the current disclosure is directed to a method of inhibiting the internalization of a targeted extracellular protein wherein the targeted extracellular protein comprises a PAN domain.

In some embodiments, inactivating the PAN domain of the targeted extracellular protein is completed by substituting at least one of the four conserved cysteines of SEQ ID NO: 1. In some embodiments, inactivating the PAN domain of the targeted extracellular protein is completed by substituting at least two of the four conserved cysteines of SEQ ID NO: 1. In some embodiments, inactivating the PAN domain of the targeted extracellular protein is completed by substituting at least three of the four conserved cysteines of SEQ ID NO: 1. In some embodiments, inactivating the PAN domain of the targeted extracellular protein is completed by substituting at each of the four conserved cysteines of SEQ ID NO: 1. In some embodiments, inactivating the PAN domain of the targeted extracellular protein is completed by substituting at least one of the four conserved cysteines of SEQ ID NO: 2. In some embodiments, inactivating the PAN domain of the targeted extracellular protein is completed by substituting at least two of the four conserved cysteines of SEQ ID NO: 2. In some embodiments, inactivating the PAN domain of the targeted extracellular protein is completed by substituting at least three of the four conserved cysteines of SEQ ID NO: 2. In some embodiments, inactivating the PAN domain of the targeted extracellular protein is completed by substituting each of the four conserved cysteines of SEQ ID NO: 2.

In some embodiments, the inactivating of the PAN domain of the targeted extracellular protein is achieved by genome editing, which is achieved by a method selected from the group consisting of CRISPR/Cas system, Cre/Lox system, TALEN system, ZFNs system and homologous recombination.

In some embodiments, the CRISPR-mediated genome editing comprises introducing into the cancer cell a first nucleic acid encoding a Cas9 nuclease, and a second nucleic acid comprising a guide RNA (gRNA), wherein said gRNA is specific to the gene.

In some embodiments, the CRISPR-mediated genome editing comprises introducing into the cancer cell a first nucleic acid encoding a Cas9 nuclease, a second nucleic acid comprising a guide RNA (gRNA), wherein said gRNA is specific to the gene encoding a PAN domain containing protein, and a third nucleic acid comprising a template for homologous recombination.

In some embodiments, the cancer cell is selected from breast cancer, colon cancer, lung cancer, skin cancer, brain cancer, blood cancer, cervical cancer, liver cancer, prostate carcinoma, pancreas carcinoma, gastric carcinoma, ovarian carcinoma, renal cell carcinoma, mesothelioma, and melanoma.

In some embodiments, the targeted extracellular protein is associated with cancer signaling pathways. In some embodiments, the targeted extracellular protein is selected from protein kinase A, Hepatocyte Growth Factor (HGF), p104 protein, and macrophage stimulating 1 (MST1) protein. p104 protein is a cell proliferation regulator identified as a binding protein of phospholipase Cγ1. MST1 inhibits the progression of breast cancer by regulating the Hippo signaling pathway.

The specific examples listed below are only illustrative and by no means limiting.

### EXAMPLES

The steps of the method described in the various examples disclosed herein are sufficient to carry out the methods of the present disclosure. Thus, in an example, a method consists essentially of a combination of the steps of the methods disclosed herein. In another example, a method consists of such steps.

The following examples are presented to illustrate the present disclosure. The examples are not intended to be limiting in any manner.

### Example 1. PAN Domain-Carrying Proteins are Enriched in Cell Recognition and Cellular Signaling Processes

PAN domain-carrying proteins do not share a clear evolutionary or phylogenetic trajectory suggesting that this domain may have been co-opted independently by organisms to serve protein functions that are yet to be revealed. To provide evidence of its putative function, we scanned the InterPro (https://www.ebi.ac.uk/interpro/) and UniProt (https://G-LecRK.uniprot.org) databases and found 28,300 proteins across 2,496 organisms falling into 959 genera. Based on predicted cellular localization, PAN domain proteins predominantly are present in the extracellular matrix (ECM) either as cell surface receptors or ligands for cell surface receptors. Gene Ontology (GO) enrichment analyses using the 28,300 proteins revealed 39 unique GO-terms that were enriched at p<0.05. These included terms such as cell recognition (p-value=IE-30), cell communication (p-value=IE-30), proteolysis (p-value=IE-pollen-pistil interaction (p-value=IE-30), reproduction (p-value=IE-30), response to stimulus (p-value=IE-30) and response to stress (p-value=IE-30). The predominant occurrence of these proteins in the ECM and their enrichment in processes typically associated with cellular signal transduction suggested that the PAN domain may play essential roles in immune response, albeit in different pathways for divergent organisms. Based on these results, a hypothesis was formed that the PAN domain would be essential for HGF/c-MET signaling. To test this hypothesis, experiments were designed to mutate core cysteine residues and assess implications on downstream signaling cascades.

### Example 2. Core PAN Domain Cysteine Residues Modulate HGF Overall-Abundance

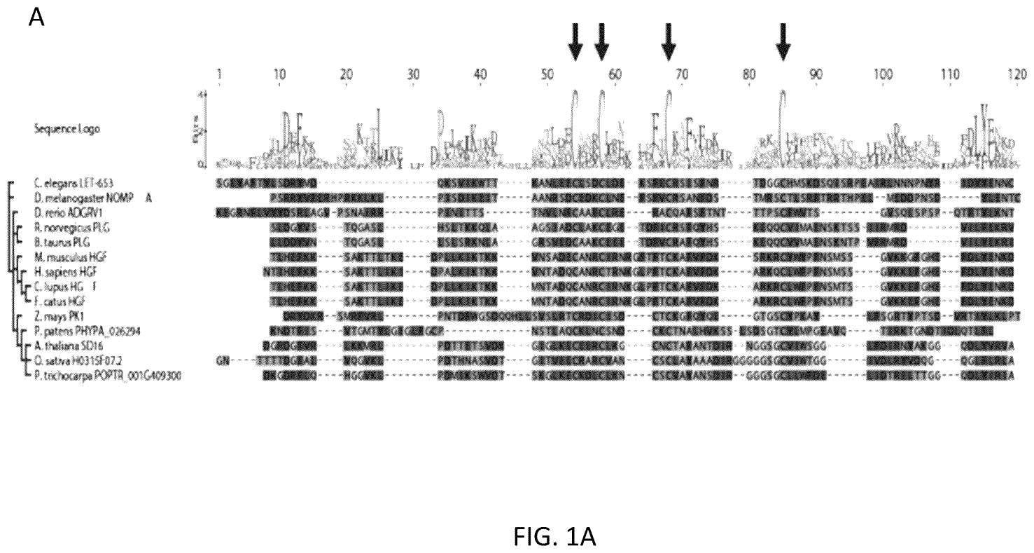

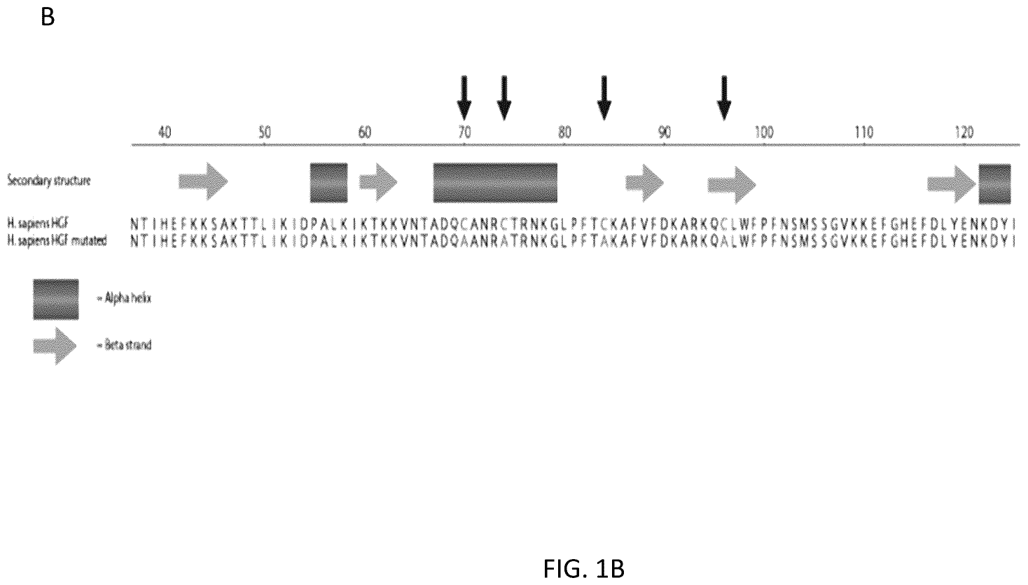

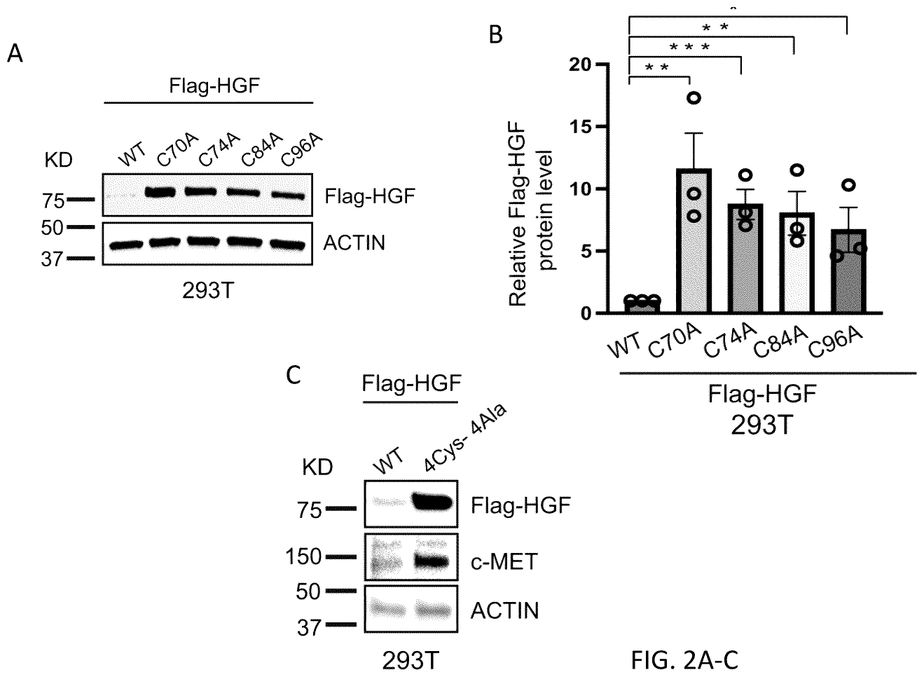

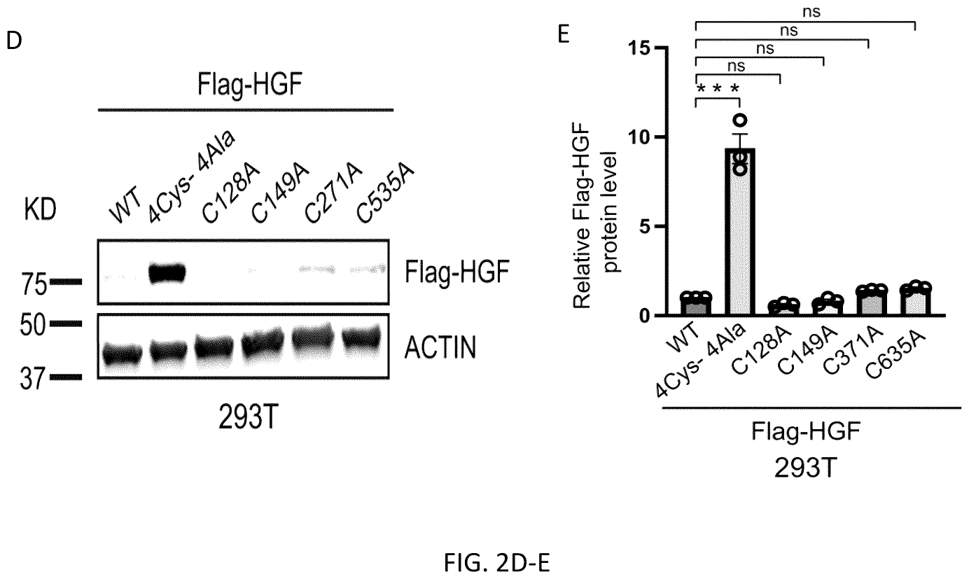

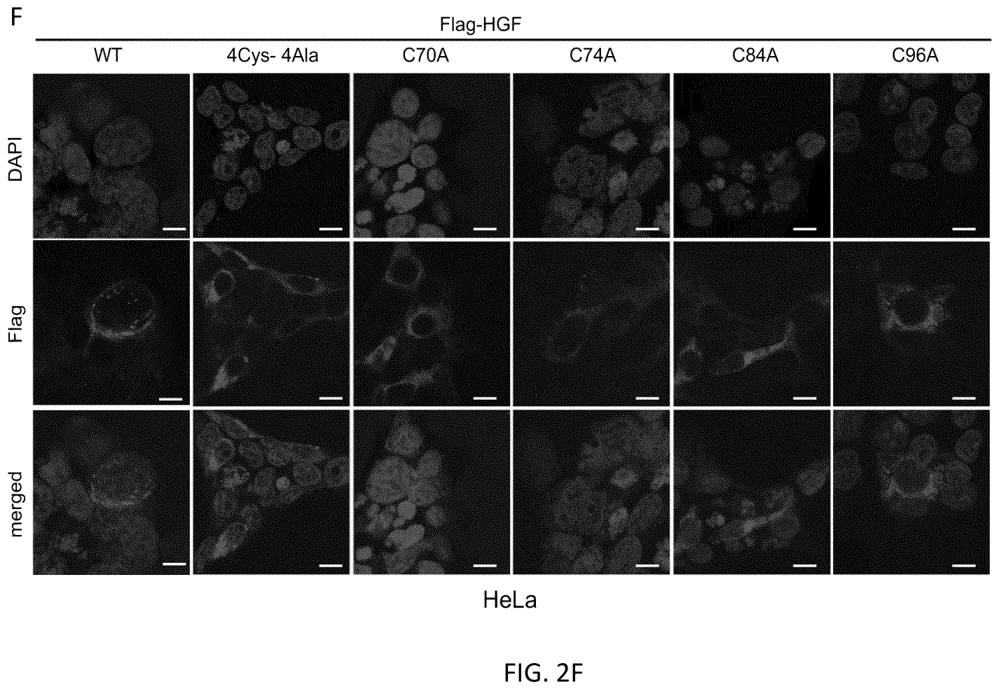

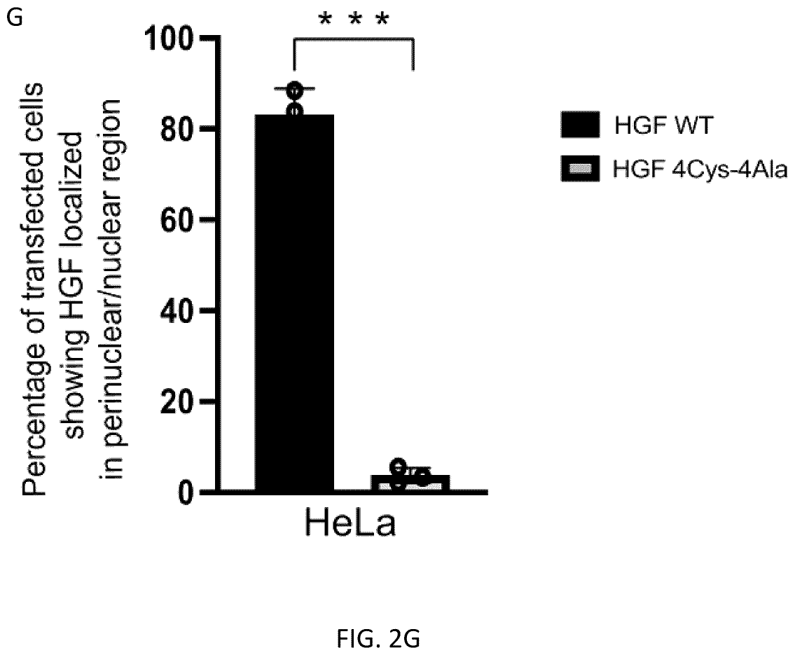

Alignment of the PAN domain of proteins from 14 model organisms revealed four strictly conserved cysteine residues occurring at amino acid positions 70, 74, 84, and 96 of the HGF protein (FIG. 1A&B;). To evaluate the functional significance of these residues, we sequentially mutated single cysteines and also simultaneously mutated all four cysteine residues and examined HGF stability in 293T cells. Mutant HGFs, in which cysteines 70 (C70A), 74 (C74A), 84 (C84A) and 96 (C96A) were substituted with alanine, led to a marked increase in the protein expression of exogenously expressed 1-IGFs in cells compared to their wild-type (WT) counterpart (FIG. 2A-B). Furtheintore, mutating all four core cysteines in the PAN domain dramatically increased the abundance of exogenously expressed HGF-4Cys-4Ala in the cells (FIG. 2C). Moreover, the c-MET receptor exhibited a similar increase in abundance in response to stimulation by the HGF 4Cys-4Ala mutant compared to WT HGF. These results suggest that mutant HGF and its c-MET receptor did not undergo the expected lysosomal degradation when the core cysteines were mutated.

The kringle domains of HGF have been reported to be crucial for protein-protein interactions. In particular, the first and second kringle domains are especially important for the proper biological function of the protein. Kringle domains are usually disulfide crosslinked domain and previous studies have established that kringle domains in the α-subunit and the SPH domain in the ˜-subunit provide c-MET binding sites on HGF. Recently, Uchikawa et al. determined structures of c-MET/HGF complex mimicking their active state at 4.8 A resolution using cryo-electron microscopy. They identified multiple distinctive c-MET binding sites on the HGF protein including the N-terminal and kringle domains but did not implicate the PAN domain in the HGF/c-MET interaction. Here, we exclusively investigated the role of the PAN domain of 163 HGF on the c-MET signaling cascade.

As we established a connection between the core cysteines in the PAN domain of HGF with HGF abundance, we introduced four more mutations of additional cysteines located in the K1, K2, and SPH domains of HGF (C128A, C149A, C271A, and C535A). None of these mutant HGFs showed increased protein expression when expressed exogenously in cells as did the HGF 4Cys-4Ala (FIGS. 2D and 2E). Thus, cysteine residues in the kringle and SPH domains are apparently not involved in HGF abundance. Rather, induced expression of HGF is specifically defined by the four strictly conserved cysteine residues in PAN domain. 100731 It was hypothesized that enhanced HGF expression of the 4Cys-4Ala mutant may results from an intra-PAN domain structural change that prevents I-IGF degradation. To test this hypothesis, molecular dynamics (MD) simulations of the WT and 4Cys-4Ala PAN domains were performed using models generated with AlphaFold2 and compared the resulting structural ensembles. Five models were generated for each system and used as starting structures for the simulations. A 200-ns simulation was then performed for each model. Thus, the cumulative simulation time was 1 is for each system. Analysis of the time evolution of the root-mean-square deviation (RMSD) of each system revealed only minor structural deviations in both sets of simulations. Using the top WT model as a reference structure, the average RMSDs and standard errors of the means were 1. 61+/−0.02 Å for the WT and 1.71+/−0.01 Å for the 4Cys-4Ala system, indicating highly similar overall structures despite the four Cys substitutions in the mutant. The structural analysis indicates that no large-scale structural changes occurred in the 4Cys-4Ala mutant PAN domain compared to the WT on this time scale.

Previous findings have suggested that, upon binding with its receptor, MFT, HGF internalization and degradation precedes activation of MET signaling. Translocation of MET-bound HGF toward the perinuclear region is a key step for the degradation of HGF and recycling of the receptor. The existence of a parallel pathway has also been reported in which HGF-activated MET translocates to the nucleus to initiate calcium signaling. In agreement with a role for the PAN domain in HGF abundance, it was found that mutating the core cysteine residues reduced the perinuclear signal for all mutated HGFs in HeLa cells (FIG. 2F). HeLa cells transfected with HGF 4Cys-4Ala showed less than 5% of the total perinuclear staining of recombinant HGF. However, under the same transfection efficiency 80% of cells transfected with WT HGF showed perinuclear staining for HGF under confocal microscopy (FIG. 2G). As such, both biochemical and immunofluorescence results suggest a critical role for core PAN domain cysteine residues in HGF stability and cellular uptake. Because all single cysteine mutants yielded the same results as the 4Cys-4Ala mutant, all subsequent studies were performed using only the recombinant HGF 4Cys-4Ala variant.

### Example 3. PAN Domain Cysteine Residues are Essential for c-MET, AKT and ERK Phosphorylation

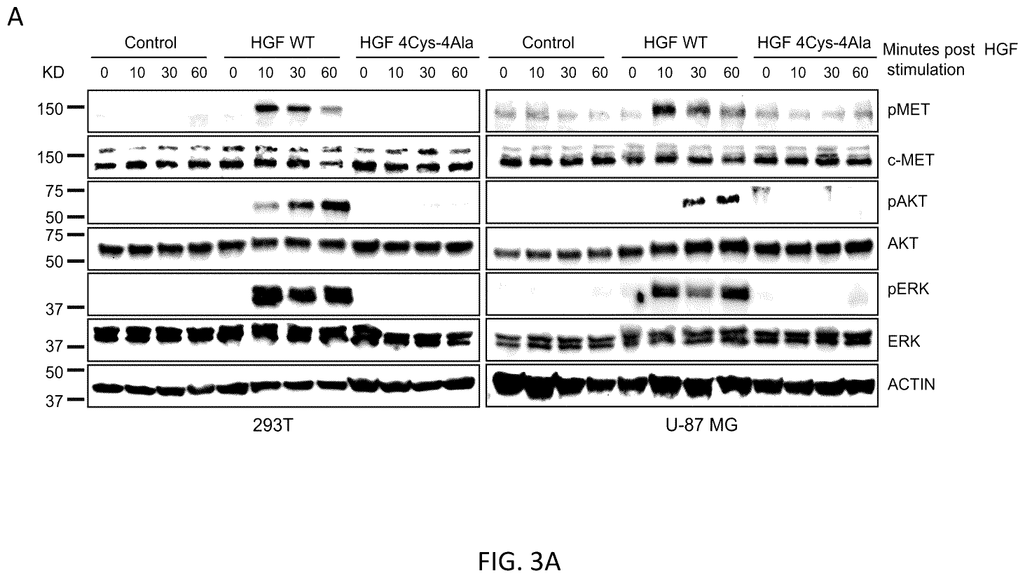

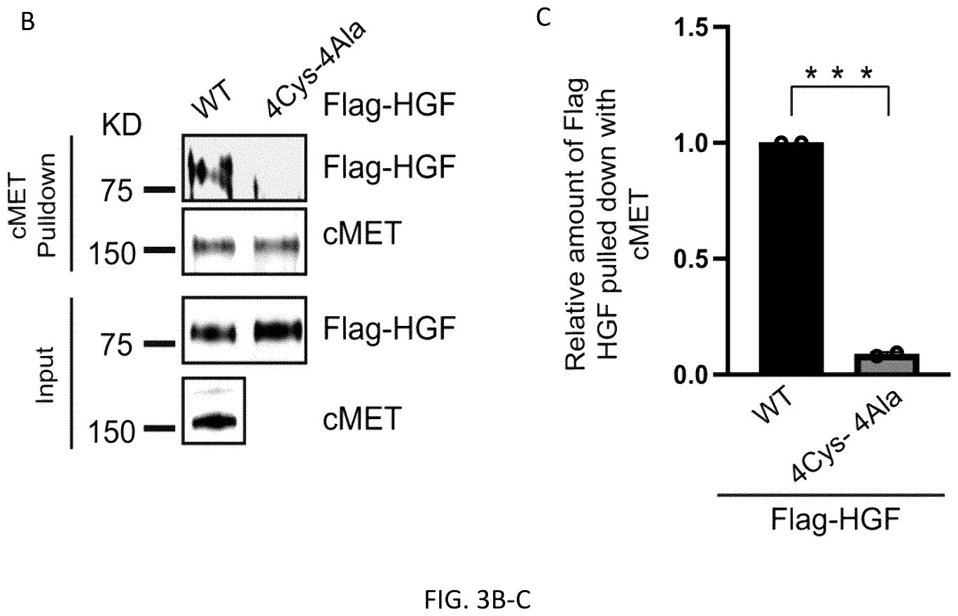

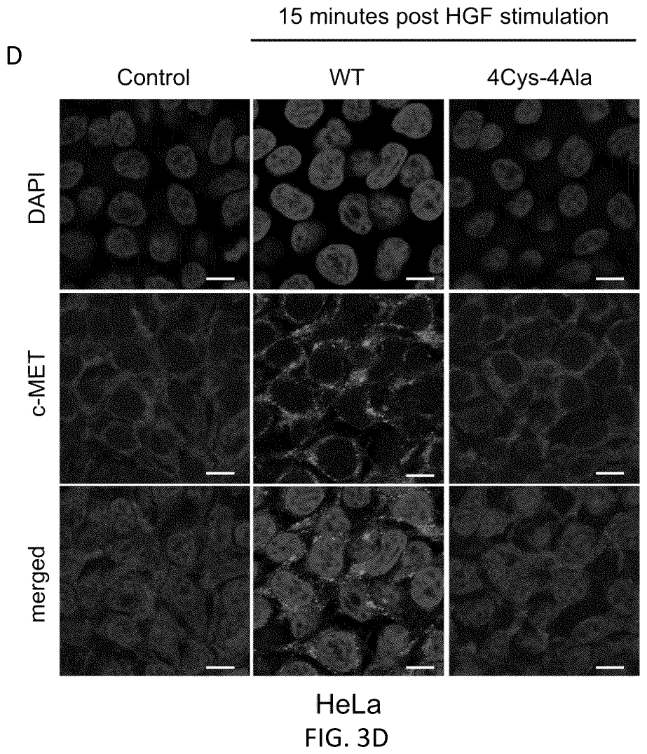

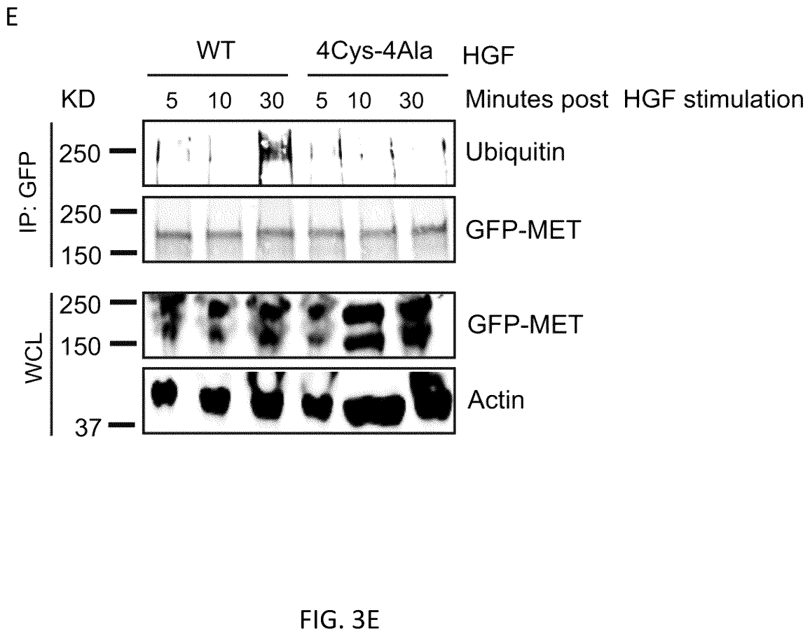

Following binding of HGF at the MET semaphoring homology (SEMA) domain, MET homodimerizes and autophosphorylates at two tyrosine residues (Y1234 and Y1235), followed by subsequent phosphorylation of two additional tyrosines in the carboxy-terminal tail (Y1349 and Y1356), This series of events creates a multifunctional docking site for downstream adaptors and effectors that lead to the activation of the Rat sarcoma (RAS)/ERIC and phosphatidylinositol 3-kinase (PBK)/AKT axis via phosphorylation. To determine the influence of core cysteines on MET phosphorylation, both 293T and glioblastoma U-87 MG cells were stimulated with purified wild-type HGF and HGF 4Cys-4Ala proteins. Western blot analyses revealed the absence of phosphorylated MET, AKT, and ERK in unstimulated controls and in cells stimulated with the HGF 4Cys-4Ala protein in both cell types (FIG. 3A).

To characterize the incompetence of PAN mutant HGF in turning on the phosphorylation cascade of c-MET, we performed a direct interaction assay between endogenous c-MET and in-vitro translated HGF proteins and showed that HGF 4Cys-4Ala was unable to interact with c-MET (FIGS. 3B and 3C). Our results thus far indicate a central role for the PAN domain in its initial recognition by c-MET. In addition, HGF 4Cys-4Ala, like the unstimulated controls, failed to promote MET perinuclear translocation compared to wild-type HGF based on stimulation assays using HeLa cells (FIG. 3D).

To gain additional insight into the impact of core cysteines in the HGF PAN domain on MET degradation, we performed an in-vivo ubiquitination assay with c-MET. Ubiquitination was inhibited with the alanine mutants of HGF compared to wild type (FIG. 2E). Based on these observations, we propose that MET receptors are unable to become internalized by endocytosis, suggesting that the mutated cysteine residues impose an overall retardation of MET activity and its endocytic trafficking.

### Example 4. Mutating Core Cysteine Residues Suppresses STAT3 Phosphorylation and Nuclear Translocation

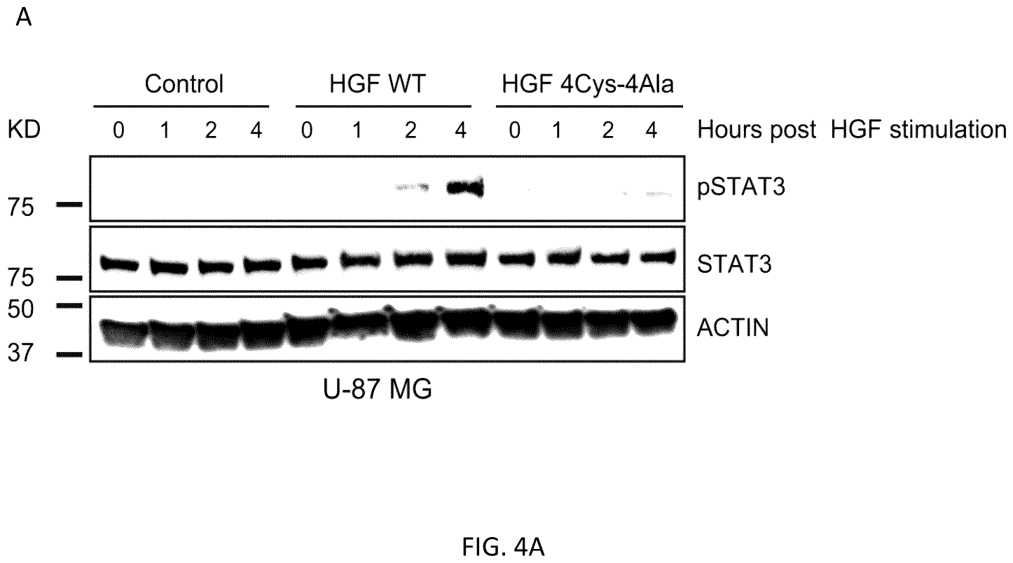

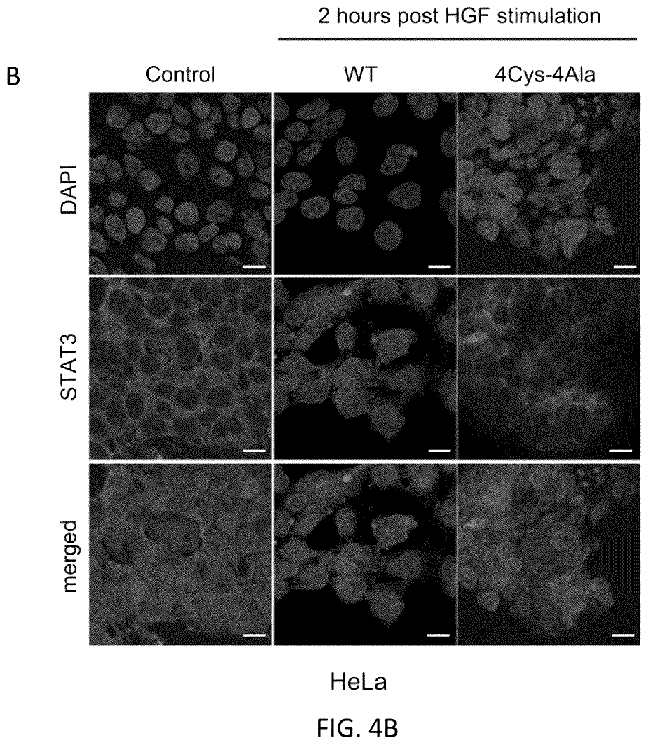

STAT3 is a transcription factor that is present in the cytoplasm, forms dimers upon activation, and functions as a downstream effector molecule of the HGF/c-MET signaling pathway. STAT3 is reported to be constitutively active in several cancers, which leads to malignant transformation by playing a critical role in stimulating cell proliferation and arresting apoptosis. The impact of the core cysteines in the HGF PAN domain on STAT3 activation was tested by following its phosphorylation in U-87 MG cells. STAT3 phosphorylation was significantly reduced in cells post-stimulation with the HGF 4Cys-4Ala mutant compared to the wild type (FIG. 4A). Delayed time points were chosen for this assay because HGF induces delayed STAT3 phosphorylation. Confocal imaging confirmed that HGF 4Cys-4Ala, like the unstimulated controls, was unable to promote STAT3 nuclear translocation, while wild-type HGF promoted normal STAT3 nuclear localization (FIG. 4B). These results are consistent with the above observations, suggesting that mutating the cysteine residues in the PAN domain leads to disrupted HGF/c-MET signaling.

### Example 5. PAN Mutations Downregulate HGF/c-MET-Dependent Cell Proliferation and Alters Expression of Genes Essential for Diverse Cellular Responses

Dysregulated expression of HGF/c-MET acts as a catalyst in many cancers, with overexpression of HGF often leading to aberrant cell proliferation and extracellular matrix invasion. Direct evidence has been established connecting a primary role for HGF with increased expression of MMP9, which is crucial for angiogenesis. HGF facilitates MMP9 expression via the PBKIAKT and p38 mitogen-activated protein kinases (MAPK) axis. Given the reported role of HGF in modulating expression of MMP9, we evaluated the impact of mutated cysteine residues on its transcriptional response. To evaluate the role of PAN domain mutation on the pattern of the signature gene expression as well as cell proliferation, both 293T and U-87 MG cells were stimulated with wild-type HGF or 4Cys-4Ala HGF for 24 hours following serum starvation. Cell viabilities, as assessed by the MTT assay, were notably reduced by treatment with HGF 4Cys-4Ala, whereas wild-type HGF could overcome serum starvation-mediated growth inhibition in both cell types. 293T cells transiently transfected with Flag-WT HGF showed a significant increase in cell proliferation over time compared to cells transfected with Flag-HGF 4Cys-4Ala. Quantitative real-time PCR (qRT-PCR) was performed to determine whether MMP9 expression was similarly impacted. mRNA levels of MMP9 were markedly decreased in HGF 4Cys-4Ala-stimulated 293T and. U-87 MG cells compared to the wild type in both cell types. Similarly, a significant decline in MET mRNA expression in both cell types stimulated with HGF 4Cys-4Ala was seen. Increased c-MET expression might be a crucial determinant for the overall balance of the HGF/c-MET cascade in cells and could be a trigger for the malignant transformation of normal cells. These data suggests that induction of the MMP9 receptor is correlated with overall MET expression in an HGF-dependent manner and could be regulated by minimally mutating the four core cysteines in the PAN domain of HGF.

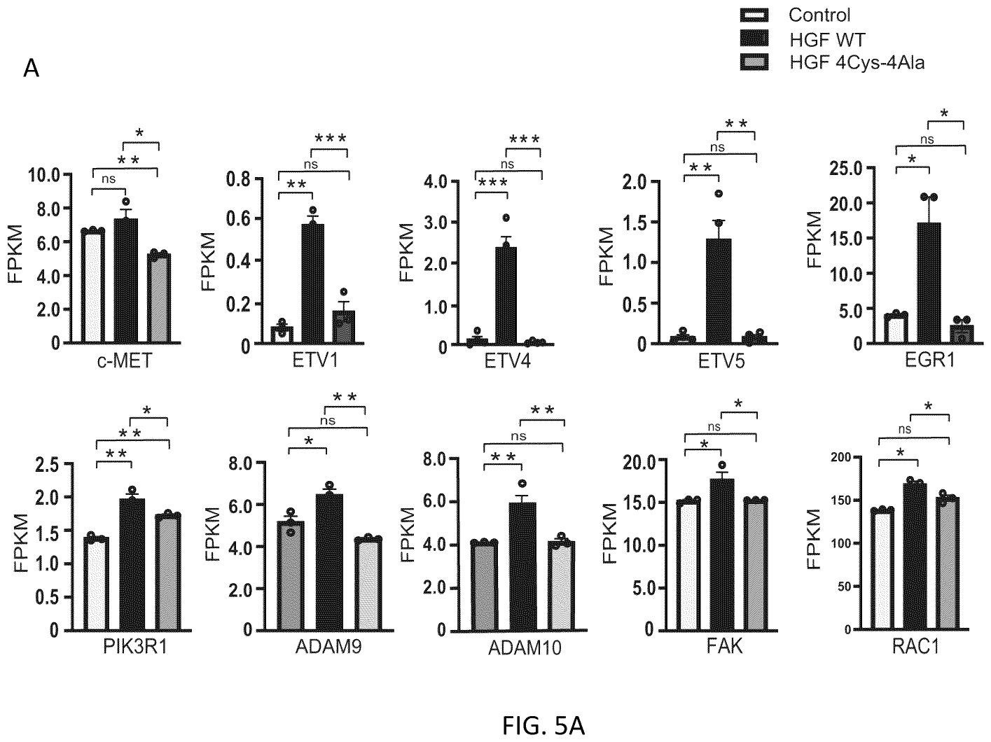

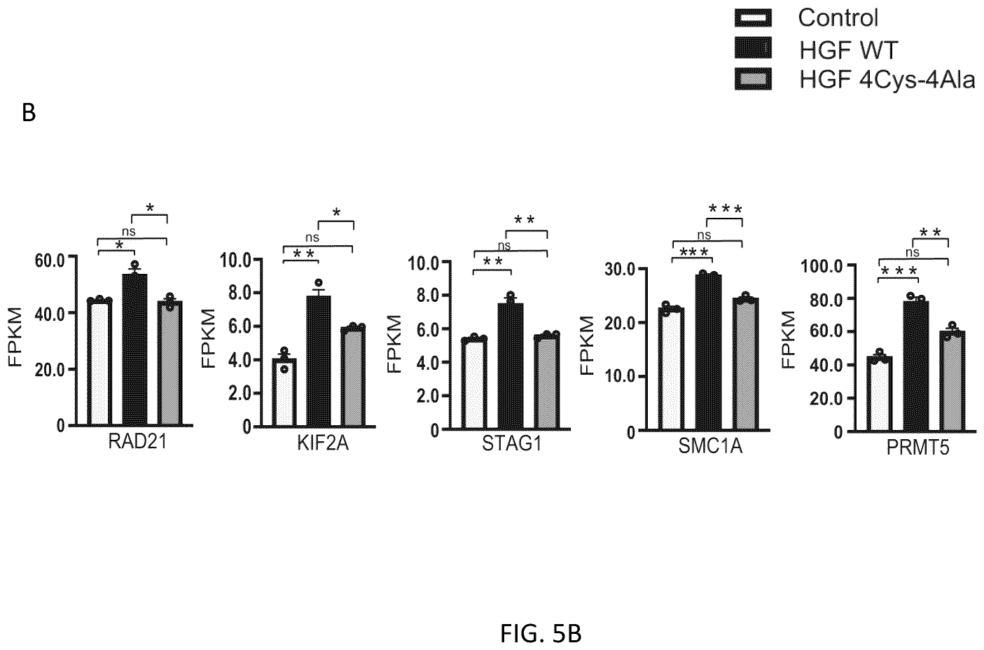

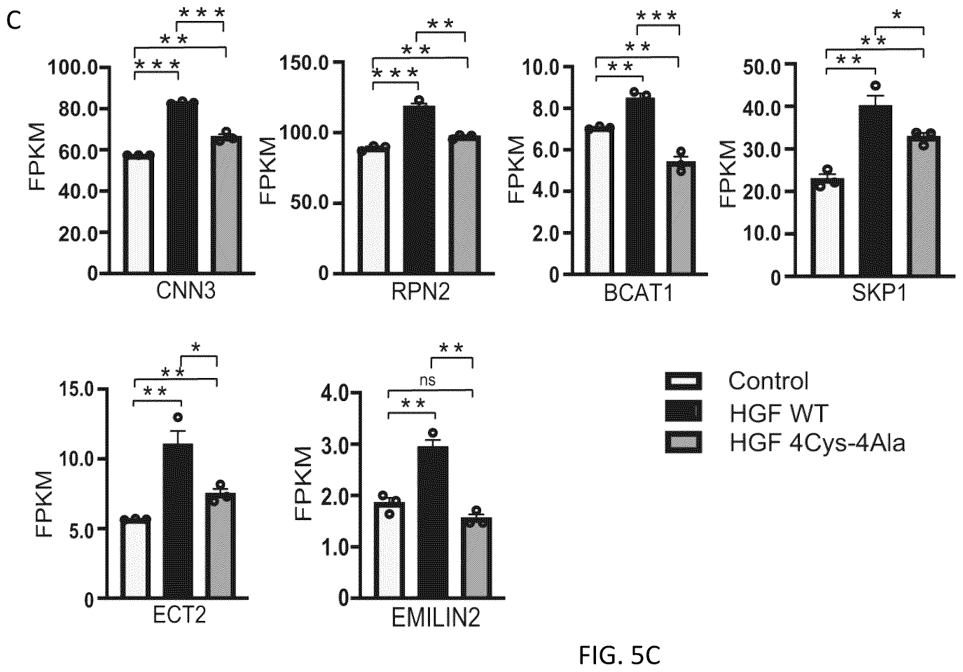

Based on the apparent PAN domain-dependent transcriptional modulation of MMP9 expression by HGF, the transcriptional responses of additional downstream genes in the HGF/′c-MET signaling pathway were characterized. Total RNA was extracted from 293T cells following a 24-hour post treatment with wild-type HGF or 4Cys-4Ala HGF. Based on this analysis, significant differences were identified in the expression of genes previously implicated in MET signaling, cell cycle regulation, and cancer-related processes (FIG. 4). Specifically, there was a significant reduction in the expression levels of the downstream targets of the MET signaling cascade, ETV1, ETV4, and ETV5, in cells stimulated with HGF 4Cys-4Ala compared to wild type HGF. These transcription factors are members of the polyoma enhancer activator 3 (PEA3) subgroup of the E-twenty six (ETS) family and confer resistance to early growth factor response (EGFR) targeted therapy in lung cancer. Some known target genes of PEA3 are matrix metalloproteinase-2 (MMP2), matrix metalloproteinase-7 (MMP7) and MMP9, which are well recognized for their role in the invasiveness of cancer. Mutating the core cysteine residues in the PAN domain led to reduced expression of EGR1, as well as the metalloproteinases, A disintegrin and metalloprotease domain-containing protein 9 and 10 (ADAM9 and ADAM10), all of which were previously linked to increased adhesion and cancer progression including, hepatocellular carcinoma, and triple negative breast cancer via the AKT/NF-K13 axis. Hyperactivation of focal adhesion kinase (FAK) and PBK/receptor for activated C kinase 1 (RAC 1) are responsible for metastasis progression and mutations of the core cysteines in the HGF PAN domain also resulted in their reduced expression. Apart from these examples, relative expression of proteins involved in DNA damage repair (double-strand-break repair protein or RAD21), chromosomal integrity and cohesion (structural maintenance of chromosomes protein IA or SMC1A), cell cycle progression (kinesin family member 2A or KIF2A), posttranslational modification, and RNA splicing (protein arginine N-methyltransferase 5 or PRTM5) were also reduced (FIG. 5). Furthermore, this analysis revealed well-known cancer biomarkers whose expression levels were affected by mutating the core cysteine residues. These biomarkers included calponin-3 or CNN3 (marker for colorectal cancer), dolichyl-diphosphooligosaccharide-protein glycosyltransferase subunit 2 or RPN2 (which inhibits autophagy and upregulates MMP9 expression), branched chain amino acid transaminase 1 or BCAT1 (which promotes hepatocellular carcinoma and chemoresistance), ski interacting protein or SKIP, epithelial cell transforming sequence 2 oncogene or ECT2 (overexpressed in different cancers including non-small cell lung cancer), and elastin microfibril interfacer 2 or EMILIN2 (which promotes angiogenesis and inflammation).

### Example 6. Conserved PAN Domain Residues Modulate WRKY33 Expression

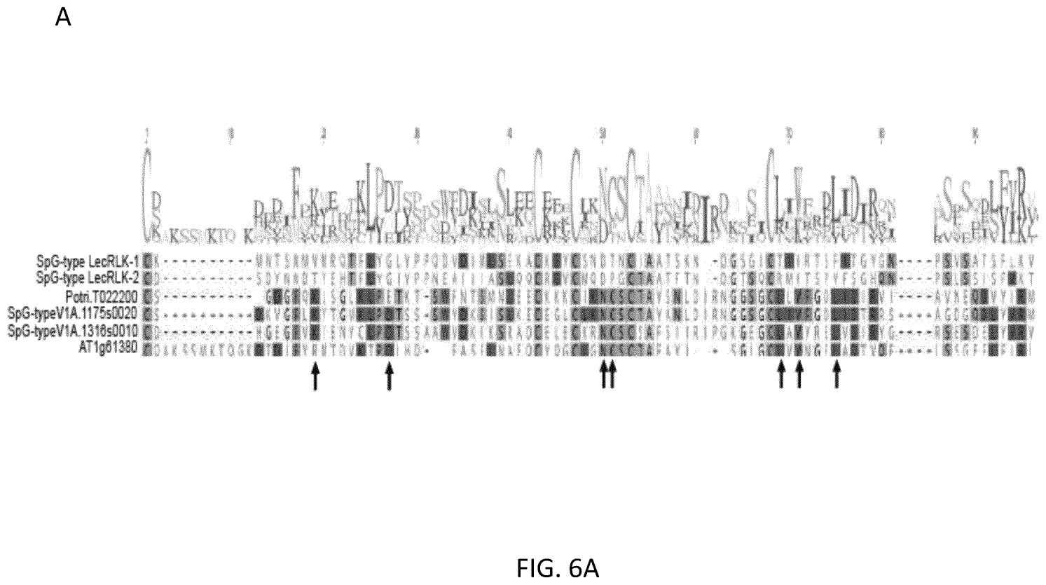

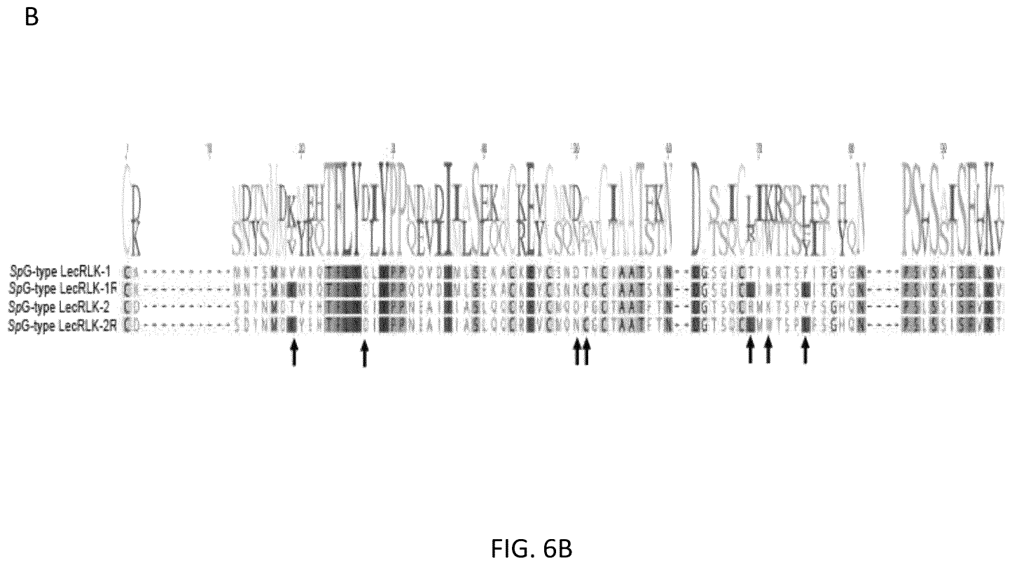

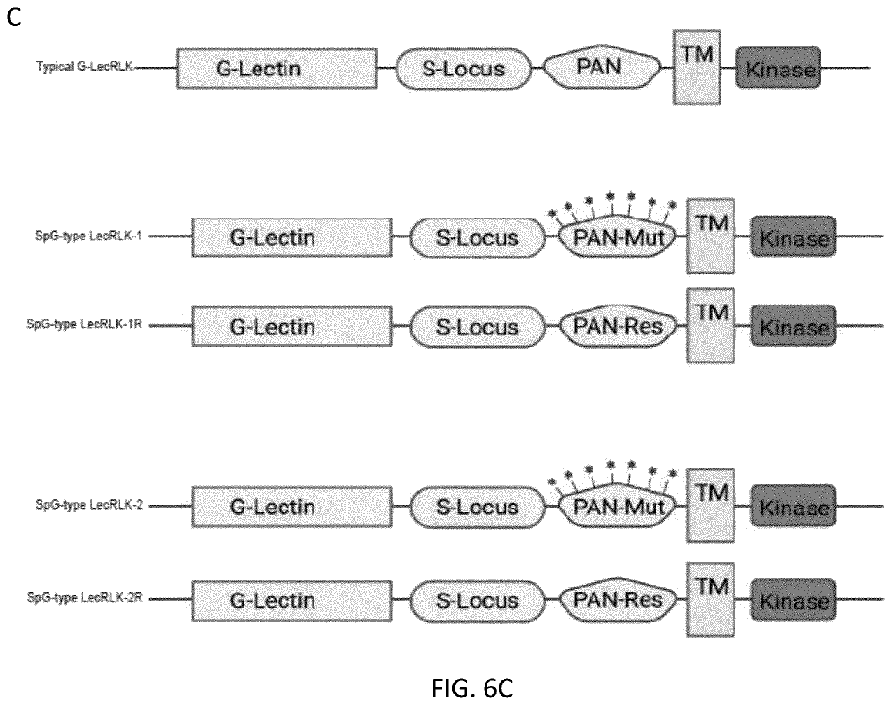

The characterization of two Salix purpurea G-type LecRLKs, SpG-type LecRLK-1 (SapurV1A.0918s0020) and SpG-type LecRLK-2 (SapurV1A.003s0270) was performed. SapurV1A.0918s0020 and SapurV1A.003s0270, unlike their previously published counterparts, are fully capable of initiating WRKY33-mediated immune responses. Protein domain prediction was performed on SpG-type LecRLK-1 and SpG-type LecRLK-2 using the InterPro database. This analysis suggested that both receptors were missing the PAN domain, which is uncharacteristic for this protein family. SpG-type LecRLK-1 and SpG-type LecRLK-2 were aligned with characterized G-type LecRLKs from Populus and Arabidopsis. The alignment revealed that both receptors had sequences consistent with the PAN domain but had acquired mutations at the same seven conserved residues which resulted in statistically insignificant prediction of the domain. Specifically, SpG-type LecRLK-1 had K19V, D27G, N50D, C51T, L69T, W71K and L75F while SpG-type LecRLK-2 had K19T, D27G, N50D, C51P, L69R, W71K and L75Y mutations within their respective PAN domains (FIG. 6A). Unlike typical G-type LecRLKs which do not elicit immune signaling, these two receptors surprisingly induced expression of the signature defense-related transcription factor, WRKY33, upon expression in Arabidopsis. As a comparator, another Salix G106 type LecRLK, SapurV1A.1175s0020, was selected which has a predicted intact PAN domain. SapurV1A.1175s0020 could not induce WRKY33 expression upon overexpression in Arabidopsis suggesting that the mutated version of PAN domain could be responsible for immune activation in plants. Thus, the observation suggested that the PAN domain may play a central role in repression of immune responses in plants.

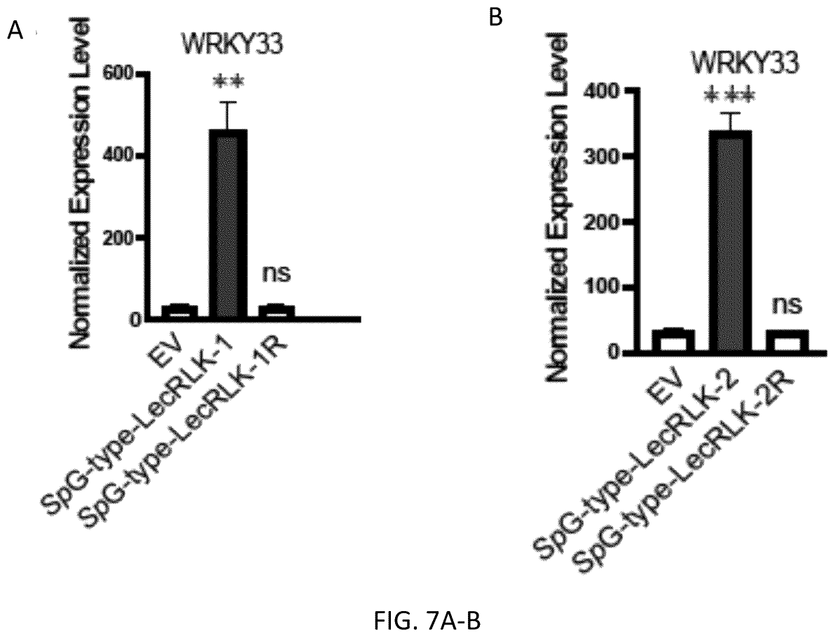

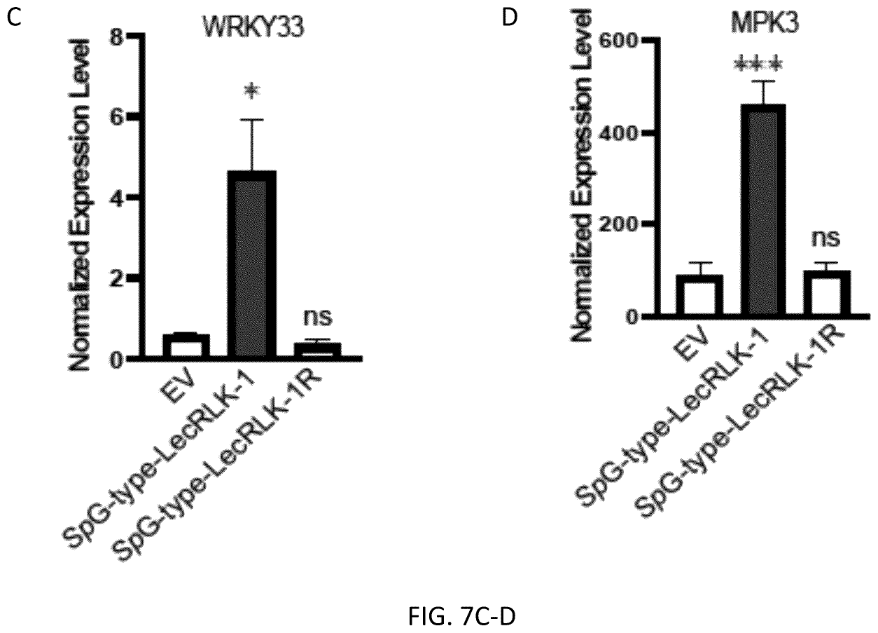

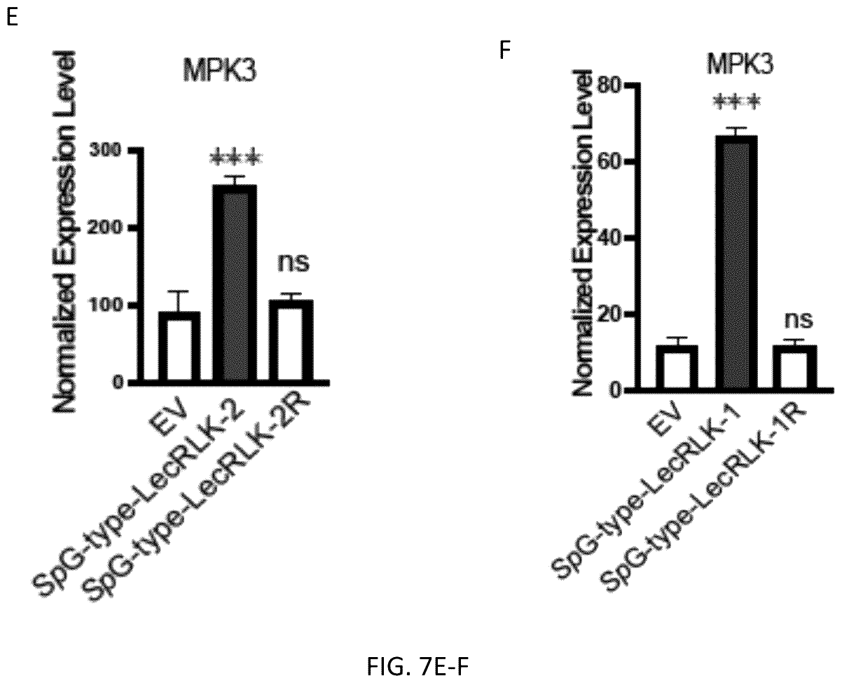

To test the hypothesis that mutated residues in the PAN domain are essential for negative defense regulation, variants were created of the two receptors with mutated amino acids restored to conserved residues and were designated SpG-type LecRLK-1R and SpG-type LecRLK-2R (FIG. 6B). Arabidopsis thaliana transgenic plants constitutively expressing the four protein variants were created for further characterization. FIG. 6C represents a schematic of typical G117 type LecRLKs, G-type LecRLKs with PAN domain mutations (SpG-type LecRLK-1 & SpG-type LecRLK-2) and G-type LecRLKs with restored PAN domains (SpG-type LecRLK-1R & SpG-type LecRLK-2R). Additionally, tobacco (Nicotiana benthamiana) expressing SpG-type LecRLK-1 and its restored version SpG-type LecRLK-1R were generated. qPCR analysis and transcriptional profiling using RNA-seq of transgenic plants confirmed our observations that SpG-type LecRLK-1 and SpG-type LecRLK-2 could significantly induce WRKY33 expression. In contrast, the two restored variants, SpG-type LecRLK-1R and SpG-type LecRLK-2R, lost their ability to induce WRKY33 expression (FIGS. 7A & C).

To investigate the subcellular localization of SpG-typeRLK-1, SpG-typeRLK-2 and its restored version, we generated constructs expressing a translational fusion of SpG-typeRLK-1 with GFP and SpG-typeRLK-1R with GFP under the control of strong cauliflower mosaic virus (CaMV) 35S promoter. Transient expression in tobacco was tested using Agrobacterium tumefaciens infiltration approach. In this experiment, both SpG-typeRLK-1 and SpG-typeRLK-1R were localized to the PM of leaf cells, although GFP alone appeared to be present in both cytoplasm and nucleus. Similarly, SpG-typeRLK-2 GFP and SpG-typeRLK-2R GFP showed a similar pattern of localization to the PM of the leaf cells. From this experiment, it was determined that there is no difference in localization patterns between the mutated and restored versions of PAN domain.

### Example 7. PAN Domain Controls MAPK Expression and Phosphorylation in Plants

It was important to assess the status of the JA/ET signaling cascades downstream of the four receptor variants. Therefore, expression of mitogen activated protein kinases (MAPKs) was evaluated. MAPKs are phosphorylation targets of the kinase domain of membrane-bound receptors. Two well-known MAPKs, MPK3 and MPK6, have been implicated in defense against pathogens. Transcriptome data from Arabidopsis transgenic plants revealed that expression of MPK3 was significantly higher in transgenic SpG-type LecRLK-1 (FIG. 7C) and SpG-type LecRLK-2 (FIG. 7E) compared to wildtype or the transgenics expressing restored variants. Similar trend we determined from tobacco transgenic SpG-type LecRLK-1 plants (FIG. 7G). Additionally, western blot analysis demonstrated that MAPK was hyperphosphorylated in Arabidopsis expressing SpG-type LecRLK-1 (FIG. 7D) and SpG-type LecRLK-2 (FIG. 7F) as well as in tobacco expressing SpG-type LecRLK-1 (FIG. 7H). However, notably reduced phosphorylation was observed in transgenics expressing restored variant (FIGS. 7D, 7F, and 7H). It was concluded that intact PAN domain suppresses pMAPK and is necessary to suppress immune activation.

### Example 8. PAN Domain Specifically Suppresses JA and ET Pathway

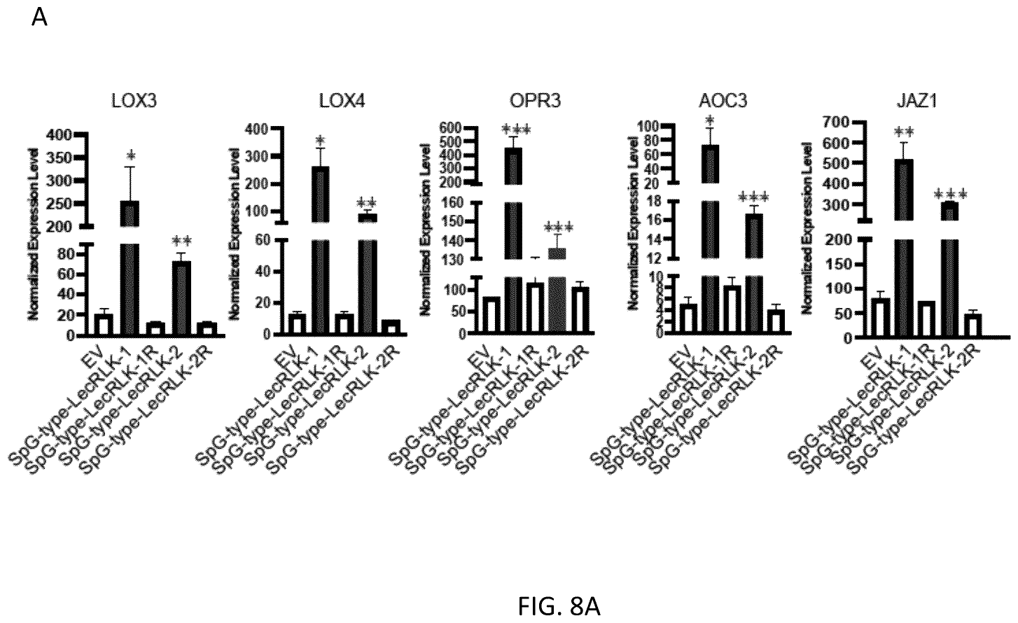

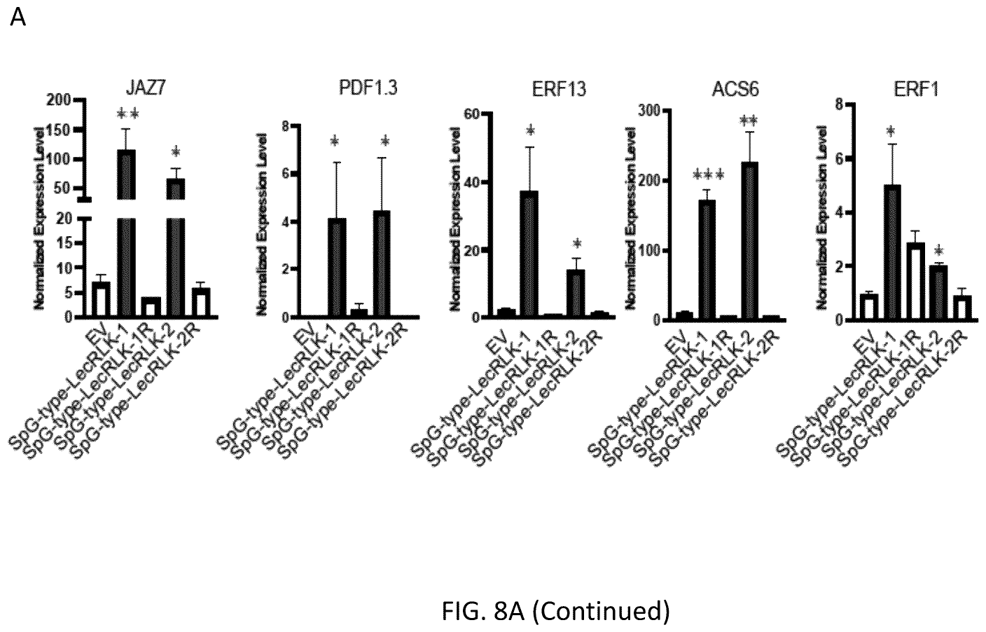

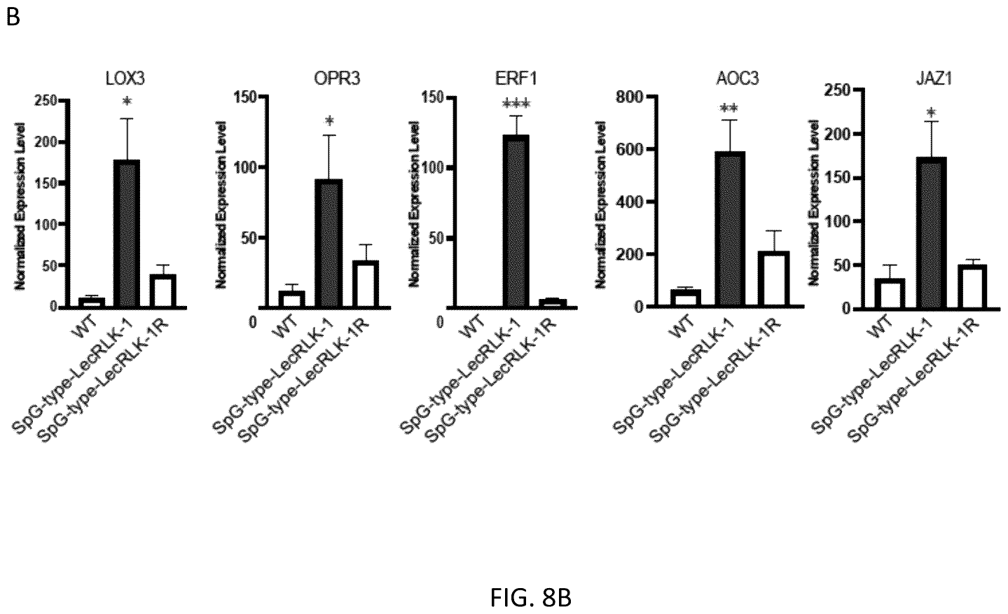

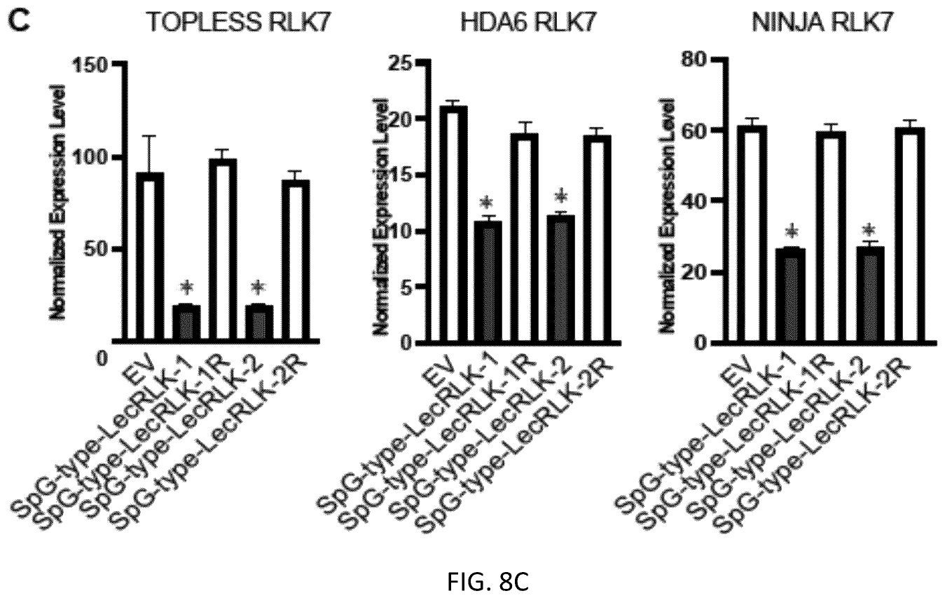

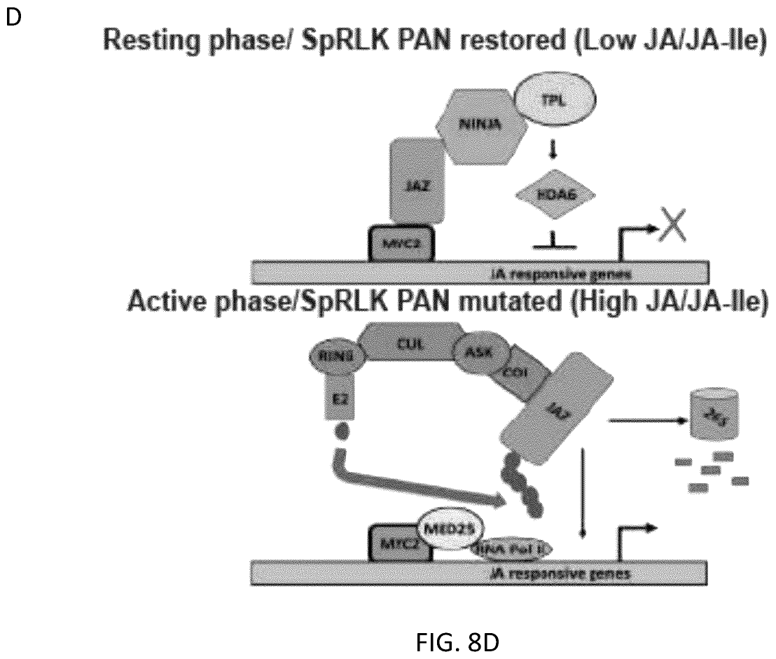

Further, expression levels of downstream genes known to mediate the JA and ET defense pathways were examined. LOX3, LOX4, OPR3, JAZ1, JAZ7, AOC3, and PDF1.3 function in the JA pathway, while ERF13, ERF1 and ACS6 function in the ET defense pathway. The expression levels of LOX3, OPR3, AOC3, JAZ1 and the ET-related transcription factor ERF1 were all assayed in tobacco. All these genes exhibited the same expression pattern and were highly upregulated in SpG-type LecRLK-1- and SpG-type LecRLK-2-expressing Arabidopsis and tobacco transgenics but showing almost wild-type levels in Arabidopsis and tobacco expressing the restored variants, SpG-type LecRLK-1R and SpG-type LecRLK-2R (FIGS. 8A and 8B).

Next, the effect of PAN domain on the transcriptional response of three well-known repressors, NINJA, HDA6 and TOPLESS, which function as negative regulators of the JA pathway. In contrast to JA and ET pathways gene above, all three repressors were significantly downregulated in SpG-type LecRLK-1 and SpG-type LecRLK-2 Arabidopsis transgenics while they were significantly higher in wildtype and transgenic plants expressing both restored variants (FIG. 8C). These results confirmed that indeed the restored variants attenuated JA signaling compared to the variants with mutated PAN domains (FIG. 8C). Expression levels of U-box proteins PUB22 and PUB23 were evaluated. PUB22 and PUB23 are known for their role in regulating amplitude of receptor-triggered immune responses at the later stages of defense signaling cascades. Expression of both SpG-type LecRLK-1- and SpG-type LecRLK-2 resulted in increased induction of PUB22 and PUB23 (data not shown). In contrast, restored variants did not induce their expression confirming that restored variants suppress defense signaling resulting in no observable immune responses (data not shown). JA and ET have also been shown to function antagonistically to abscisic acid (ABA) signaling. So, expression levels of OST1, VSP1, and VSP2 were evaluated. OST1 is a serine threonine protein kinase and, along with VSP1 and VSP2, is a known positive regulator of the ABA signaling pathway. All three showed reduced expression in SpG-type LecRLK-1 and SpG-type LecRLK-2 transgenic lines while their expression was remarkably higher in wildtype and transgenics expressing restored variants SpG-type LecRLK-1R and SpG-type LecRLK-2R (data not shown).

Global differential expression analysis showed that 6,931 genes were differentially expressed in SpG-type LecRLK-1 when compared to empty vector control. However, it was observed that a significantly reduced number of genes (1,515) that were differentially expressed between the restored variant, SpG-type LecRLK-1R compared to empty vector control. Similarly, for SpG-type LecRLK-2, 6,942 genes were differentially expressed compared to controls whereas 1,078 genes were differentially expressed in the restored variant SpG-type LecRLK-2R compared to empty vector control. GO enrichment analysis revealed that terms related to response to wounding, response to chitin, and regulation of jasmonic acid were enriched among differentially expressed genes. These results support the hypothesis that the PAN domain plays a crucial role in repression of immune response in plants.

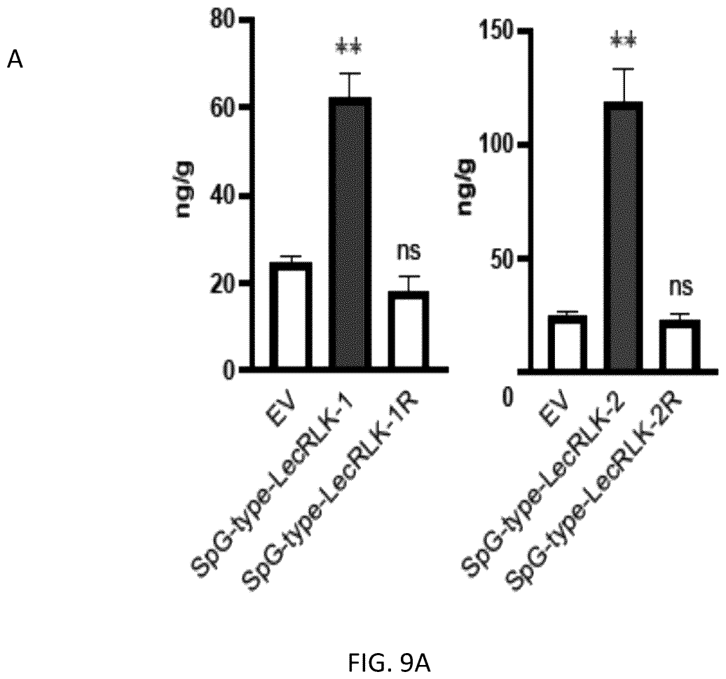

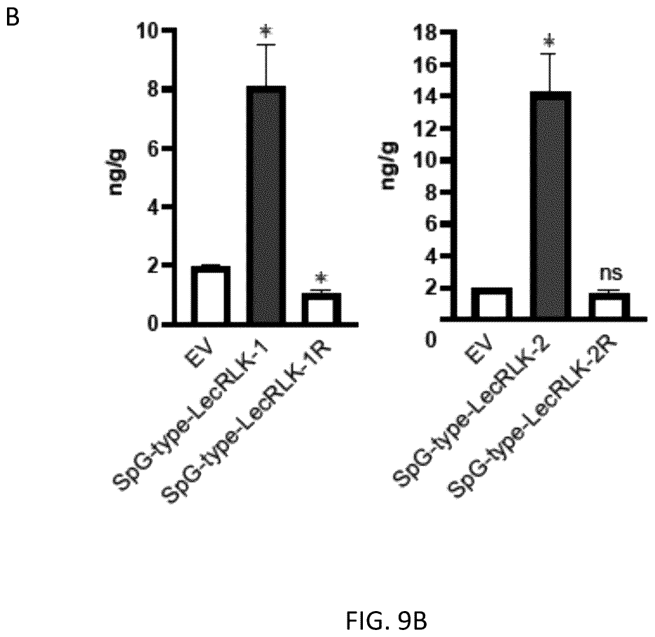

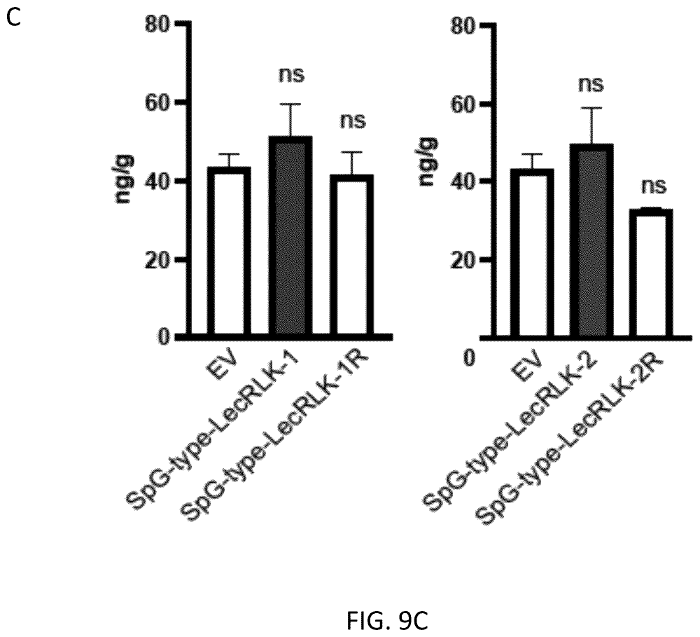

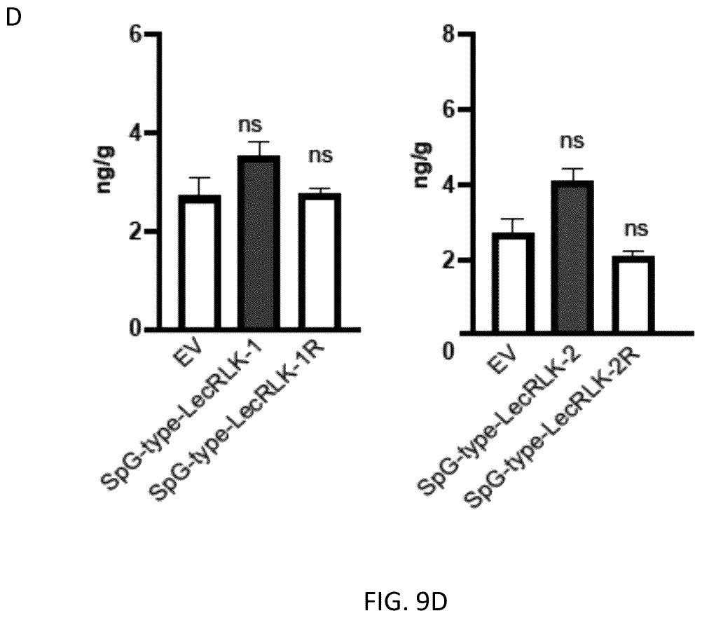

Phytohormone analysis was performed and JA, jasmonoyl-isoleucine (JA-IIe), (SA) and (ABA) levels were measured in Arabidopsis to confirm differences in signaling between SpG-type LecRLKs with mutated PAN domain amino acid residues and their restored variants. Consistent with results of the transcriptome studies, JA and JA-IIe hormone levels were significantly higher in SpG-type LecRLK-1 and SpG-type LecRLK-2 transgenic Arabidopsis while the restored variants had empty vector control levels (FIGS. 9A and 9B). SA and ABA exhibited negligible differences across empty vector control and all transgenics expressing the four variants (FIGS. 9C and 9D).

Taken together the qPCR, RNA-seq, MAPK phosphorylation and hormone analyses described above demonstrate that minimal mutations that accumulated in the PAN domains of SpG-type LecRLK-1 and SpG-type LecRLK-2 were sufficient to interfere with their negative regulatory activity and restore JA and ET immune responses. Restoration of these mutations to their conserved amino acids reconstituted negative regulation of JA and ET immune signaling, suggesting an essential role of the PAN domain in this phenomenon.

### Example 9. PAN Domain Modulates ROS Response and Immune Suppression to Botrytis cineara

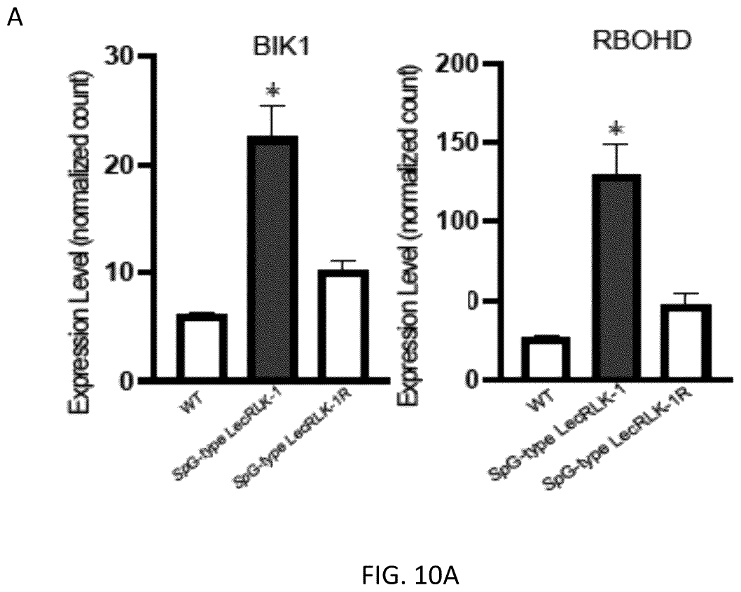

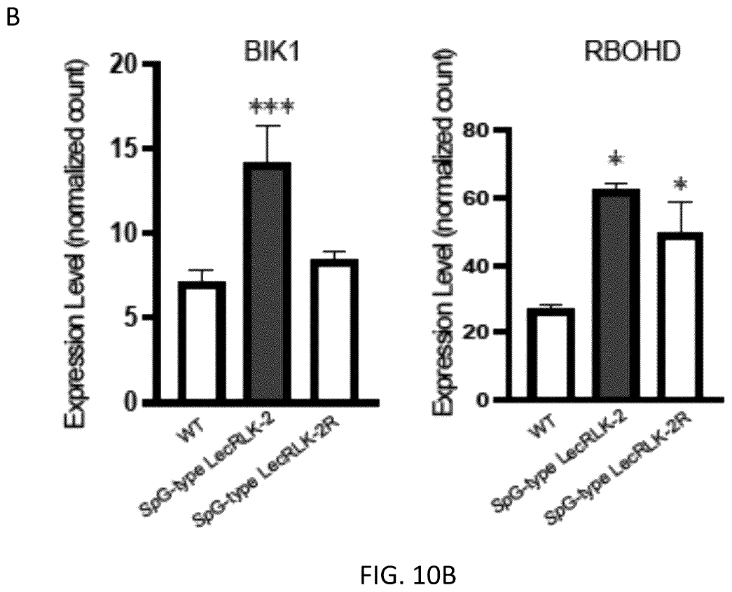

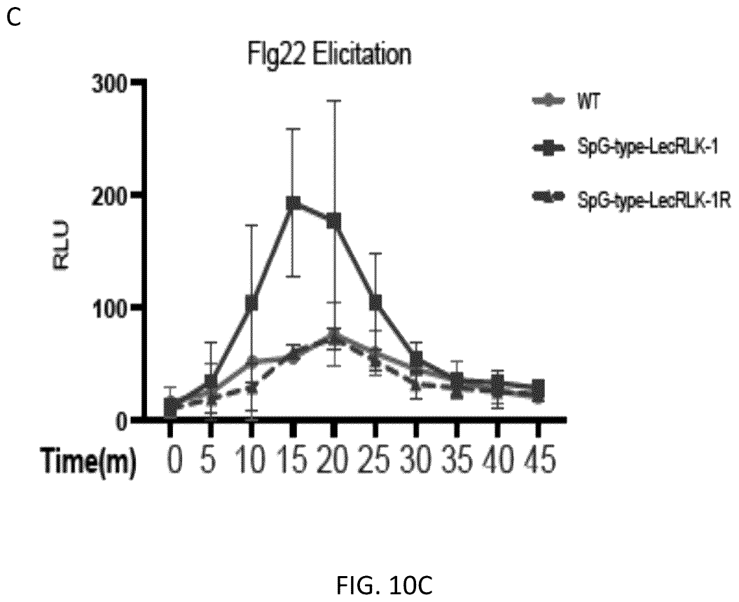

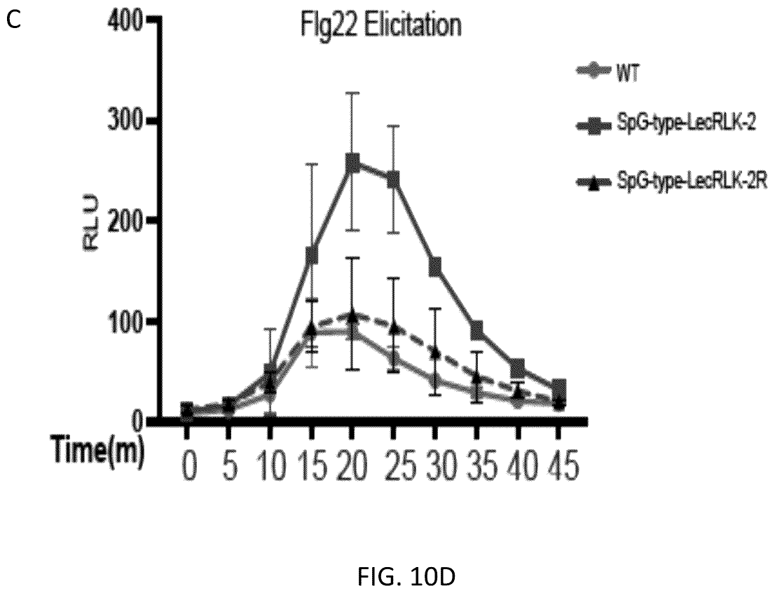

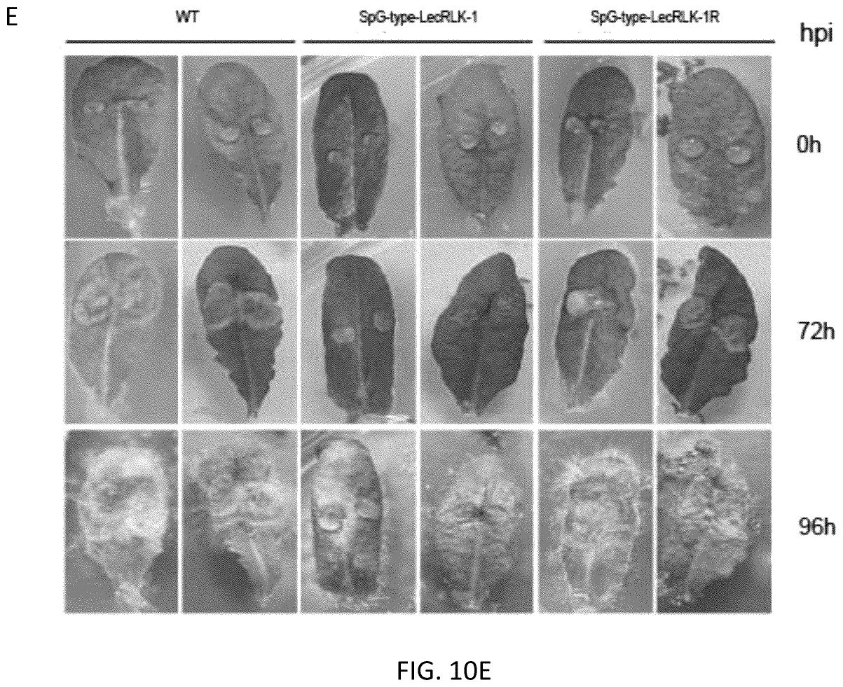

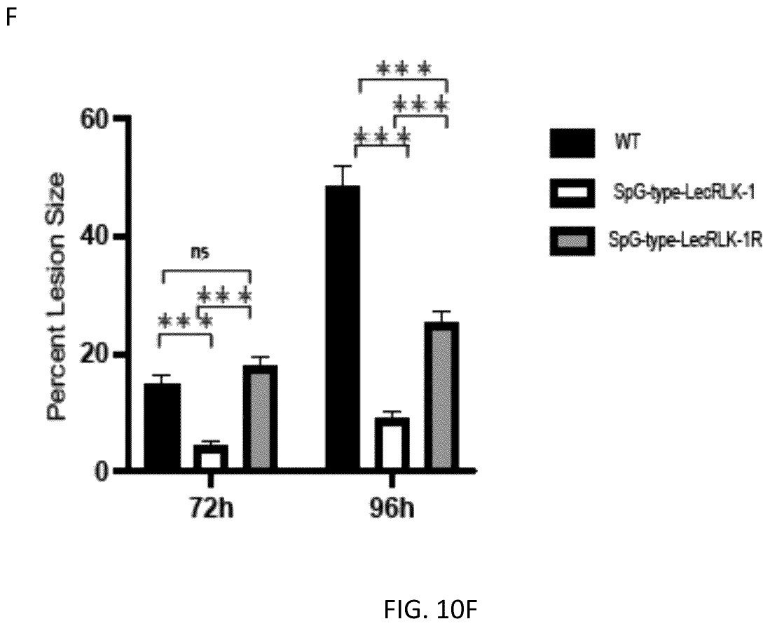

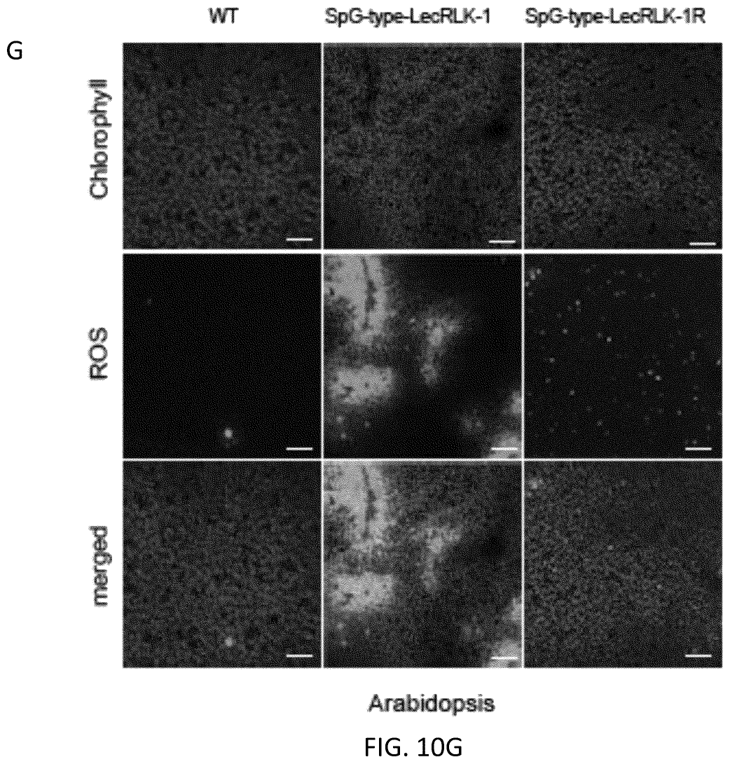

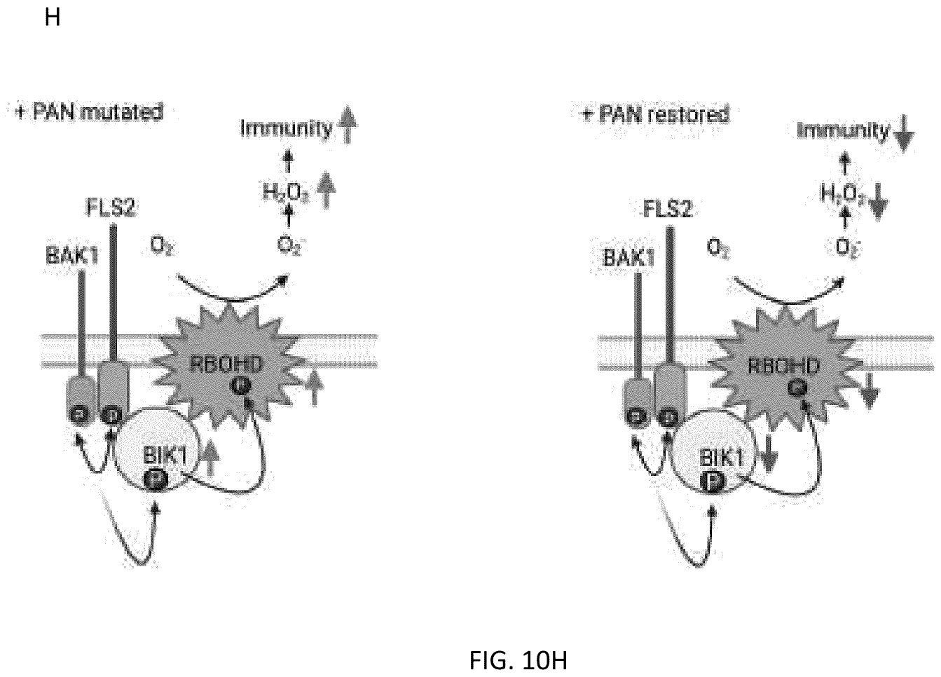

Given the cumulative observations above, we sought to establish 217 functional consequences on defense against the pathogen, Botrytis cinerea. The receptor like cytoplasmic kinase Botrytis induced kinase 1 (BIK1) serves as a central regulator during PAMP-triggered immunity in plants. Phosphorylation of BIK1 induces its stability and activation, which in turn activates respiratory burst oxidase homolog protein D (RBOHD). Activation of RBOHD releases reactive oxygen species (ROS) which is essential for plant immunity. Transcript levels of BIK1 and RBOHD were higher in both SpG-type LecRLK-1 and SpG-type LecRLK-2 transgenics compared to wildtype (FIGS. 10A and 10B). As shown in FIGS. 10C and 10D, the oxidative burst triggered by the perception of flg22 was measured in a luminol dependent assay. This analysis showed that SpG-type LecRLK-1 and SpG-type LecRLK-2 transgenic lines had distinct enhancement of the oxidative burst compared to wildtype or transgenics expressing restored variants (FIGS. 5C and 5D). Additionally, we demonstrated activation of ROS by analyzing leaves of SpG-type LecRLK-1 transgenic lines using the dye H2DCFDA under confocal microscopy (FIG. 10G). Further, Arabidopsis transgenics expressing SpG-type LecRLK-1 showed enhanced resistance against Botrytis compared to wildtype or its restored variant. Wildtype and SpG-type LecRLK-1R transgenic leaves developed more necrosis compared to SpG-type LecRLK-1 as depicted in (FIGS. 10E and 10F). This pathogen assay conclusively demonstrated that JA and ET defense pathways were activated in SpG-type LecRLKs with mutated PAN domains but were repressed upon restoration of PAN domain amino acids to conserved residues which is further depicted by a proposed model shown in (FIG. Therefore, the PAN domain underlies the observed negative regulation of JA and ET signaling by G-type LecRLKs in plants.

### Example 10. PAN Domain Self-Interacts and Modulates Oligomerization

To provide mechanistic evidence for the basis of the observed differences between mutated SpG type LecRLKs and their restored variants, we performed biochemical experiments to establish any difference in protein behavior. Previously it has been demonstrated that the PAN domain mediated S-locus receptor dimerization in the absence of a ligand (Naithani et al., 2007). Oligomerization of receptor-like kinases has been demonstrated to be essential for the activation of receptor kinases in plants and animals (Lemmon and Schlessinger, 1994; Williams et al., 1997). Therefore, experiments were done to identify any changes in the oligomeric state of mutated and restored forms of the two receptors. In this experiment, only the PAN domains were evaluated instead of full-length proteins.

Size exclusion chromatography indicates SpG-type LecRLK-1PAN is present as a dimer or trimer/tetramer. SpG-type LecRLK-1RPAN expression was quite poor relative to its counterpart, and most of the protein aggregated, which is likely the reason for the low levels of secretion. The oligomeric profile of SpG-type LecRLK-2PAN indicated that the protein was present in the solution is roughly split between monomeric (51.8%) and a higher-order complex that is likely a trimer or tetramer (48.2%). In contrast, only 26.4% of LecRLK-2RPAN was monomeric, and most of the protein formed higher-order complexes, confirming the hypothesis that an intact PAN domain promotes receptor oligomerization.

PAN domains of both SpG-type LecRLK-1 and SpG-type LecRLK-1R were expressed with different tags, GFP and FLAG in HEK293T cells. Co-immunoprecipitation results showed that PAN domains from both proteins were able to self-interact and the degree of interaction in the restored variants was higher compared to mutated forms. These data also indicated that SpG-type LecRLK-1 and SpG-type LecRLK-2 have fewer oligomeric species compared to the restored variants.

### Example 11. PAN Domain Conserved Residues are Critical for G-Type LecRLK Stability

We were interested to evaluate the stability of the mutated and restored forms of PAN domain containing G-LecRLKs. To determine the mechanism, experiments were conducted to test the stability of the mutated and restored forms of both receptors using the cycloheximide chase assay in Arabidopsis protoplasts. Both SpG-type LecRLK-1 and SpG-type LecRLK-2 exhibited significantly extended stability over time compared to the restored variants SpG-type LecRLK-1R and SpG-type LecRLK-2R.

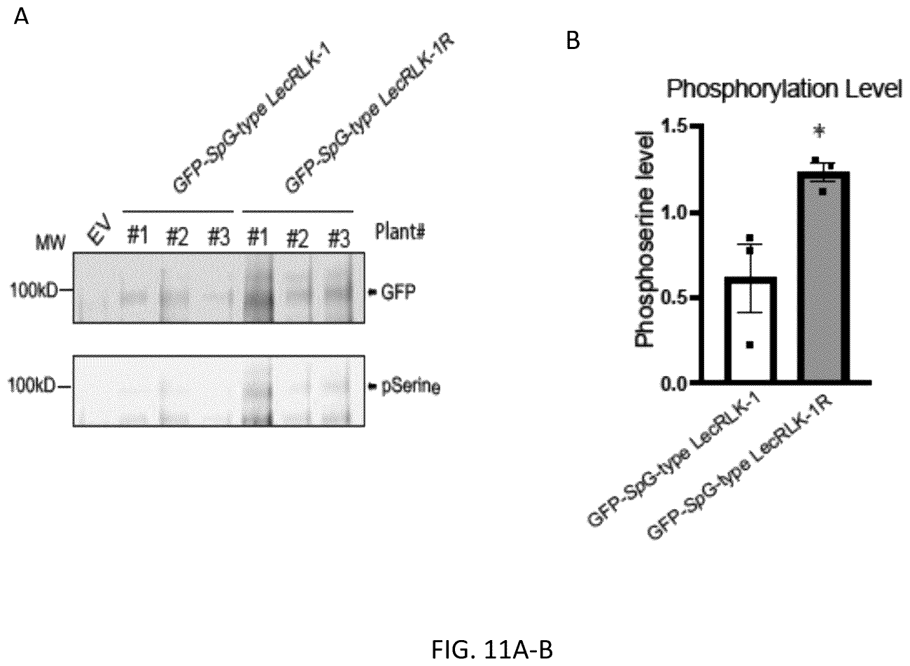

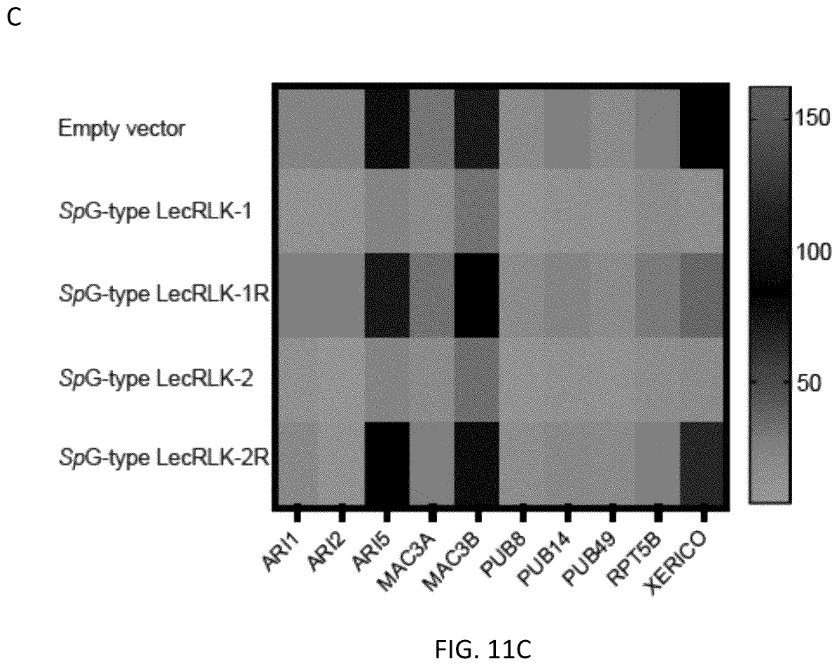

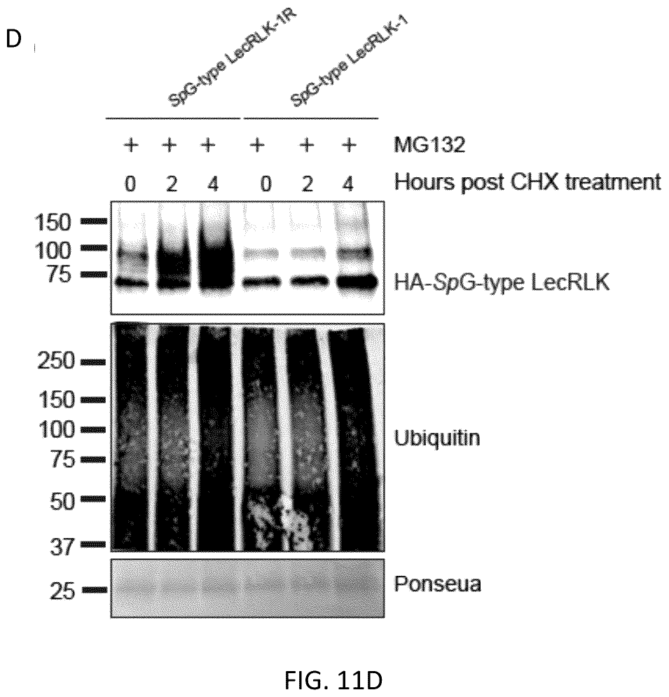

The G-Lectin RLK family includes the S-locus receptor kinase (SRK), a receptor-like kinase which is involved in the self-incompatibility in the Brassica family. Previous studies confirmed that expressing recombinant SRK proteins in insect and E. coli cells lead to autophosphorylation on serine and threonine residues. These phosphorylation events were transphosphorylation, which suggested the existence of homo-oligomers of recombinant SRK proteins. Since we also confirmed higher ordered oligomers in the restored Salix G-type LecRLKs, we sought to investigate the phosphorylation status of the PAN mutated and PAN restored SpG-type LecRLK-1. GFP-SpG-type LecRLK-1 and GFP-SpG-type LecRLK-1R were overexpressed in Arabidopsis. Western blot analysis revealed that the restored version had an increased phosphorylation level as determined by pan phosphoserine antibody (FIGS. 11A and 11B), suggesting that the PAN domain is essential for autophosphorylation of G-type LecRLKs.

Then, it was determined if there were any ubiquitin ligases involved in the degradation pathway targeting the restored variants. Transcriptome analysis from Arabidopsis transgenic lines revealed several E3-ubiquitin ligases with higher expression levels in SpG-type LecRLK-1R transgenic lines including ARI5, MAC3B and XERICO (FIG. 11C). If indeed restored variants were being targeted for degradation, it was hypothesized that MG132 which is well known proteasomal inhibitor could block the degradation of SpG-type LecRLK-1R. As expected, pretreatment with MG132 resulted an accumulation of SpG-type LecRLK-1R along with slower migration bands (FIG. 11D). Since these slower migrating bands represents poly-ubiquitinated form of SpG-type LecRLK-1R, these results suggest that an intact PAN domain in SpG-type LecRLK-1R enhances homodimerization and leads to receptor ubiquitination prior to degradation by 26S proteasome, suggesting that the PAN domain is required for proteolytic degradation of Sp G-type LecRLK-1R.

### Example 12. Conserved Cysteine Residues in PAN Domain are Critical for Immunosuppression in Plants

A characteristic pattern of the PAN domain is presence of 4-6 conserved cysteine residues that form its core (FIG. 1A). The PAN domain was implicated in protein ubiquitination and proteolysis of an oncoprotein, hepatocyte growth factor in human. Mutation of cysteine residues in the PAN domain of HGF led to major changes in HGF/cMET signaling in human cells. In addition to the HGF protein, mutating cysteine residues in a putative vestigial PAN domain in the human neuropilin protein, a receptor for SARS-Cov-2 binding and viral internalization, resulted is significantly reduced viral coat protein internalization.

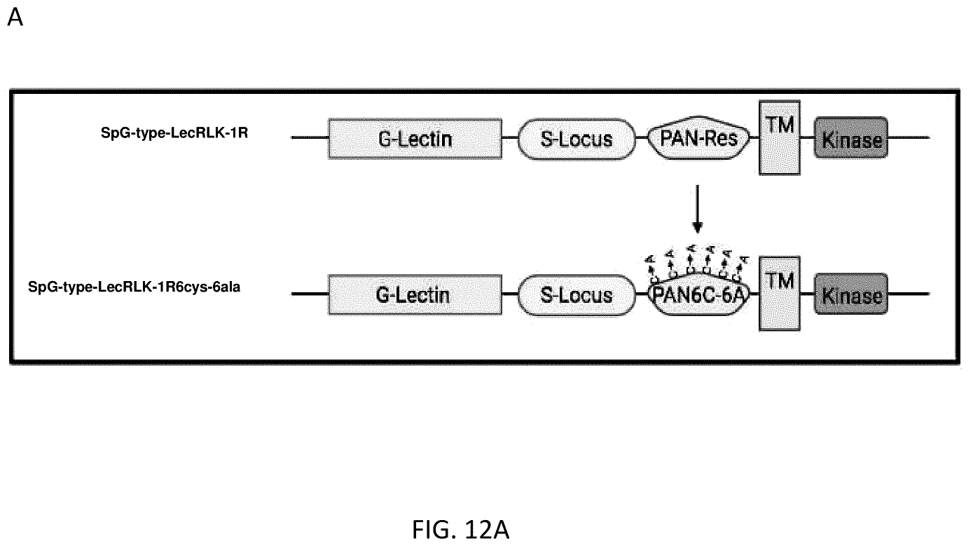

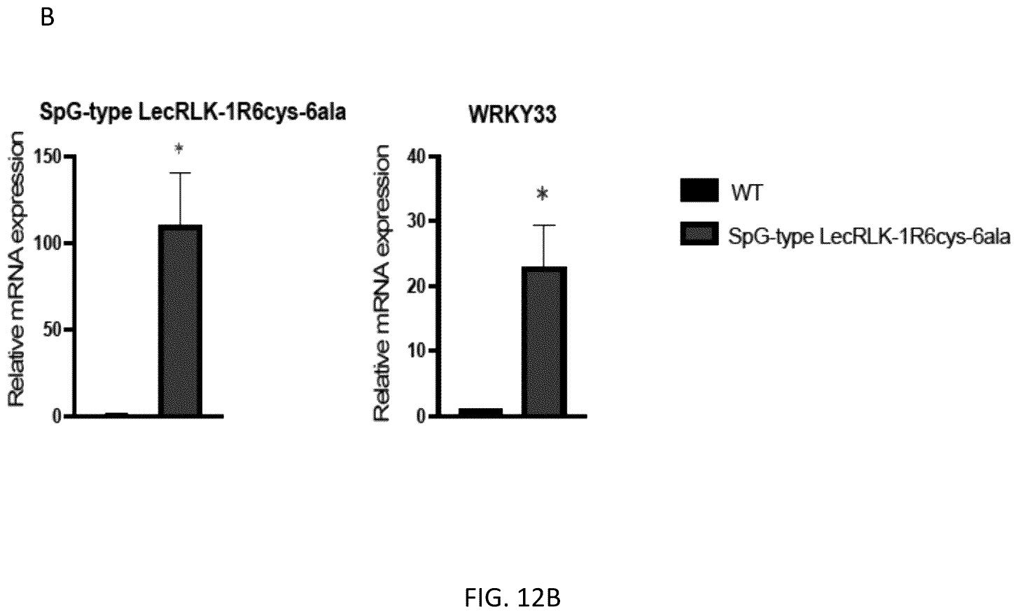

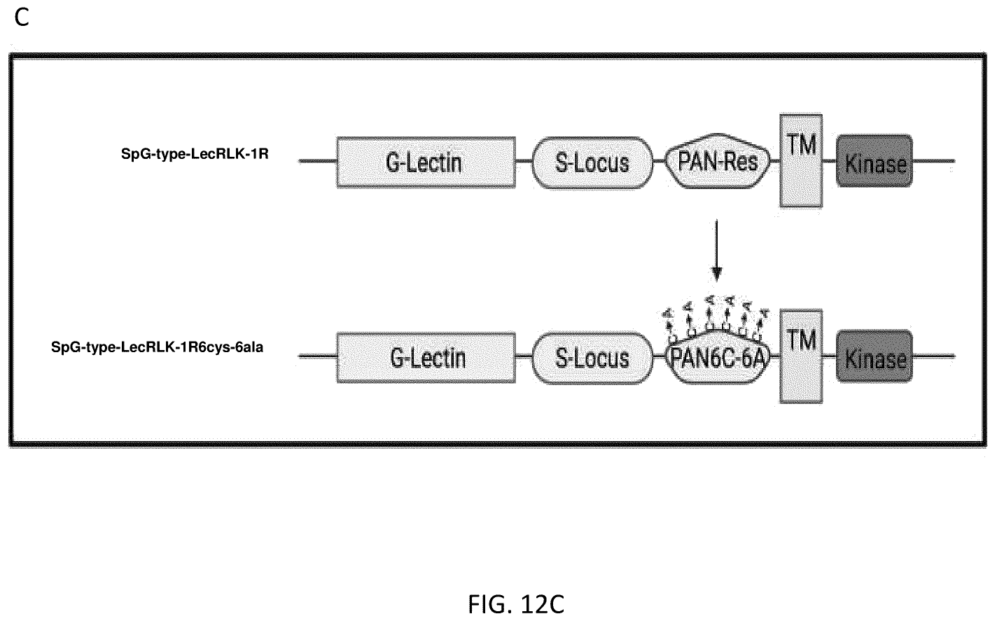

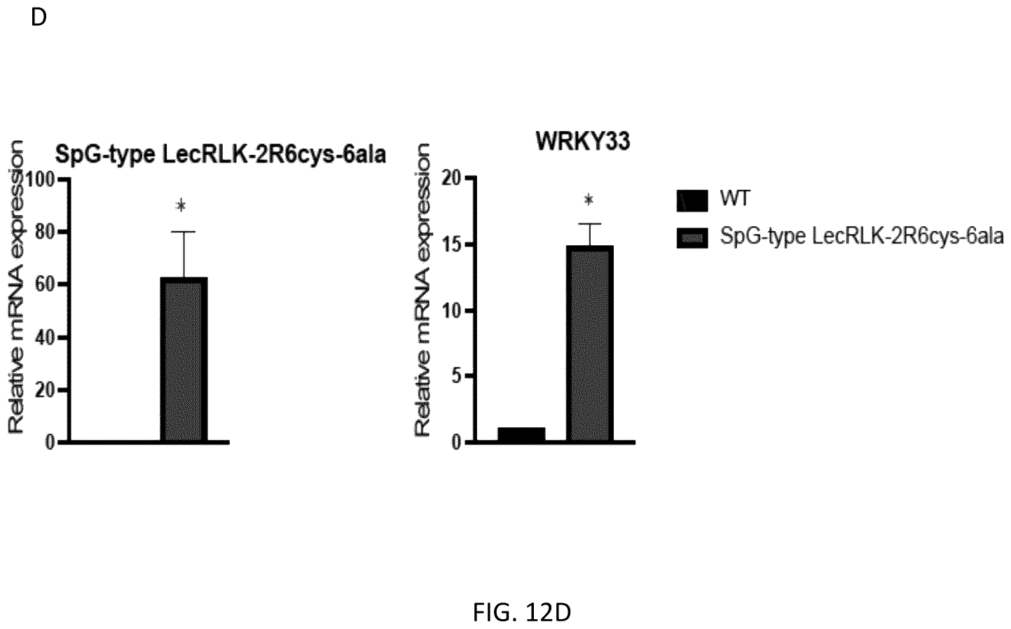

Based on these observations, we sought to evaluate the importance of conserved cysteine residues in the PAN domain of G-type LecRLK. To that end, we created 6 cysteine to 6 alanine mutation in both SpG-type LecRLK-1R and SpG-type LecRLK-2R variants (FIGS. 12A and 12C). Transgenic Sp G-type LecRLK-1PAN6cys-6Ala and SpG-type LecRLK-2PAN6cys-6ala Arabidopsis plants showed induced transcript level of WRKY33 which suggests that conserved cysteines in the PAN domain of G-LecRLK also play a crucial role in immunosuppression (FIGS. 12B and 12D).

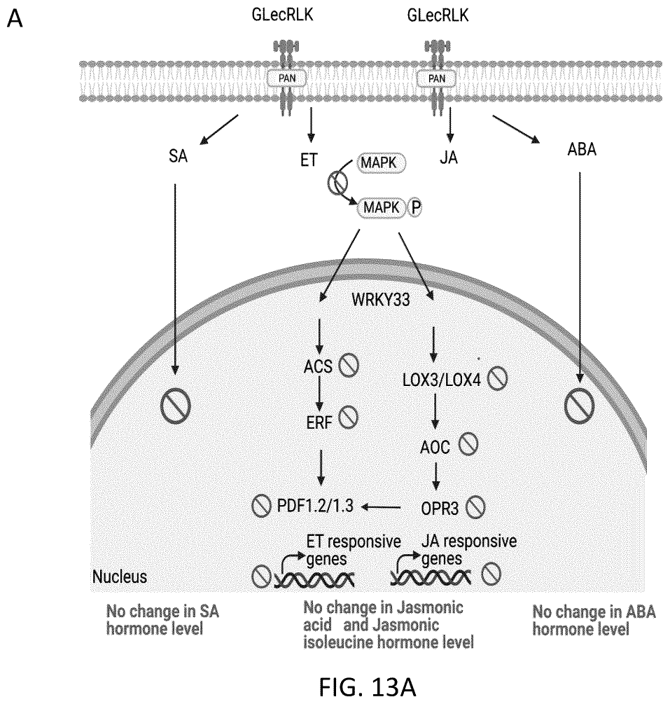

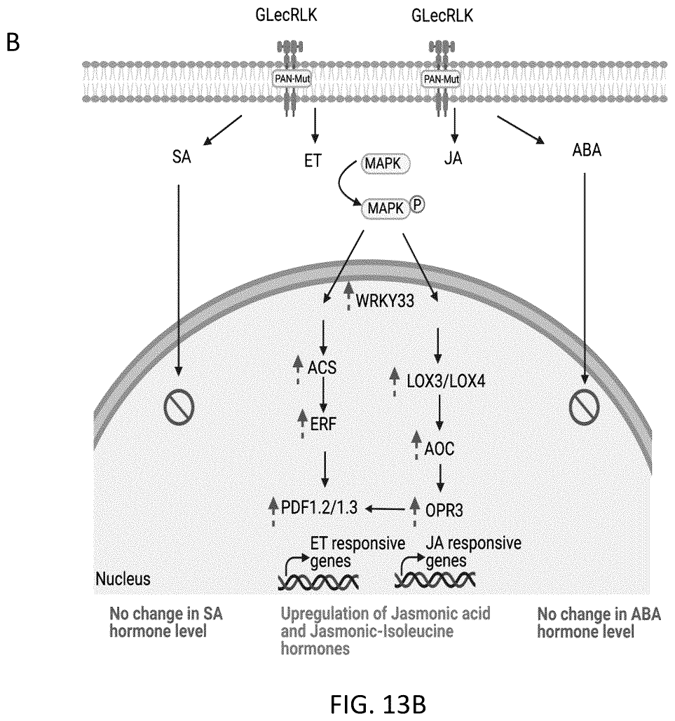

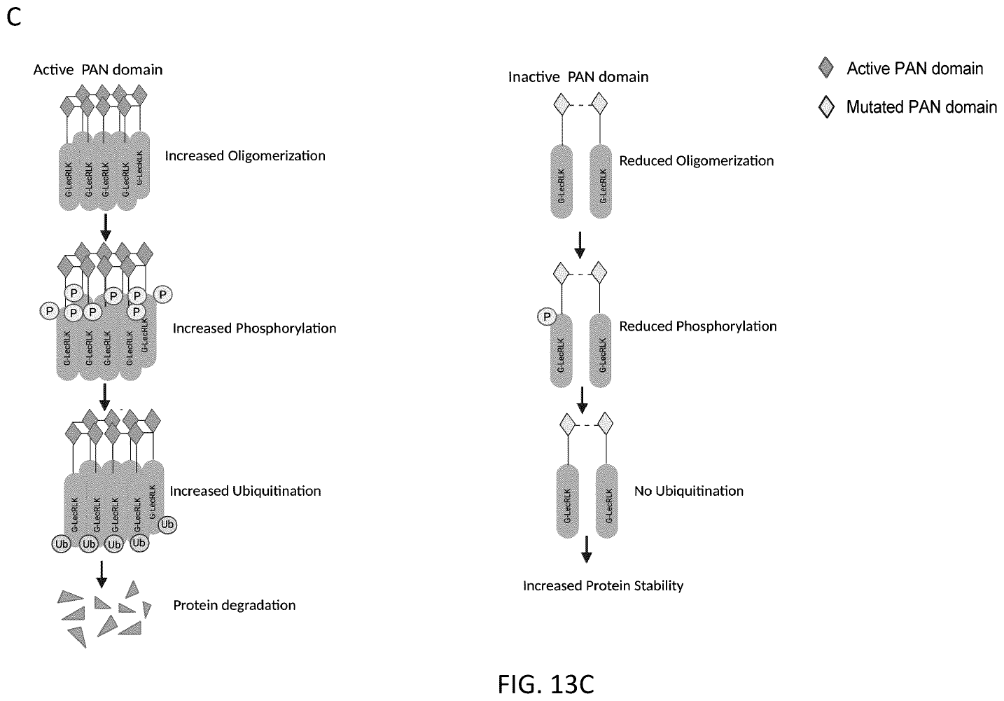

The examples herein provide evidence that the PAN domain is required for suppression of JA and ET mediated defense signaling in plants. Molecular, genetic, transcriptomic, and biochemical evidence was presented to show naturally occurring mutations in conserved amino acid residues of the PAN domain in two Salix G-type LecRLKs trigger defense signaling when expressed in Arabidopsis and tobacco. Restoration to conserved amino acid residues disrupted defense signaling. The PAN domain and its conserved amino acid residues promote suppression of JA and ET pathway by enhancing ubiquitination and proteolytic degradation of G-type LecRLKs to impair kinase activity and subsequent downstream signaling cascades (FIGS. 13A, 13B, and 13C). Mutating the PAN domain impedes receptor degradation (FIG. 13C) and restores kinase activity to trigger downstream signaling cascades and JA and ET biosynthesis (FIG. 13A).

General Methods

Mammalian Cell Culture, Transfection, and Drug Treatment

HeLa, HEK 293T, and Glioblastoma U87 cells were obtained from ATCC and maintained in a humidified atmosphere at 5% CO2 in Dulbecco's Modified Eagle's (DMEM) complete medium (Corning) supplemented with 10% fetal bovine serum (FBS; Seradigm) in 37° C. Plasmid transfections were done with TranslT-LT1 (Minis Bio) per the manufacturer's instructions.

Plant Material and Growth Condition

Arabidopsis thaliana, Columbia ecotype and Nicotiana benthamiana plants were grown in Metro-Mix 200 soil or on germination plates (Murashige and Skoog) a growth chamber at 22° C. with a 16-h-light/8-h-dark cycle. Leaves were collected from rosette-stage plants grown on soil and used for DNA isolation, and genotyping. For transcript and protein analysis all samples were harvested, frozen in liquid N2, and stored at −80° C. until needed. The coding sequences of SpG-type LecRLK-1 (SapurV1A.0918s0020), SpG-type LecRLK-2 (SapurV1A.003s0270), and SapurV1A.1175s0020 were amplified by PCR from cDNA derived from Salix purpurea leaves using HiFi polymerase (Clontech). Sequences were determined using Phytozyme using the Salix purpurea v.1.0 genome. The pENTR/D-TOPO entry vector was used for the insertion of the PCR products using the pENTR Directional Topo Cloning Kit (Thermo Fisher Scientific). LR Clonase II Enzyme Mix (Thermo Fisher Scientific) was used to transfer the coding sequences into the pGWB402-omega destination vector and transformed into One Shot TOP10 Chemically Competent cells. Vectors were transformed into the Agrobacterium strain GV3101 and transformed into Col-0 Arabidopsis thaliana using the floral dip method. Transformed seeds were selected with Kanamycin and grown in long day growth chambers at 16/8 hours light/dark at 22° C. Likewise restored versions, SpG-type LecRLK-1R and SpG-type LecRLK-2R cloned into binary vector pGWB402 omega for transformation.

Cross-Linking Assay

The hetero tri-functional cross-linker Sulfo-SBED (2-[6-(biotinamido)-2-(p-azidobenzamido)-hexanoamido]ethyl-1,39-dithiopropionate from Thermo Scientific #33073) was used for studying the interaction between HGF and c-MET per the manufacturer's instructions. Briefly, purified Flag-HGF WT or Flag-HGF 4Cys-4Ala (1 μg each) was used as a bait protein and labeled with Sulfo-SBED for 30 min at room temperature in the dark. Unincorporated cross-linker was removed by dialyzing the reaction mixture against 1× Label Transfer Buffer at 4° C. for overnight in the dark. SBED-labeled HGFs were added to the purified His-c-MET protein and incubate for 45 min. Following UV cross-linking for 15 min (6-watt hand-held lamps at distance of 5 cm), the disulfide bond of Sulfo-SBED was cleaved by 2-mercaptoethanol resulting in a biotin label attached to the interacting c-MET protein conjugate. Samples were divided into two equal parts and the biotin labeling of c-MET was analyzed by electrophoresis, followed by western blotting using Streptavidin-HRP as probe for one part and anti-His and anti-Flag antibodies for the other part. For the in-vivo cross-linking, HEK293T lysate expressing GFP-C-MET protein was substituted for purified His-c-MET and incubated at 4° C. for 45 min in the dark. After UV cross-linking (6-watt hand-held lamps at distance of 5 cm), biotin-containing complexes were immunoprecipitated with streptavidin beads (1 h at 4° C., Pierce™ Streptavidin Magnetic Beads). Immunoprecipitates were washed three times with PBS and analyzed by Western blotting on reducing (mercaptoethanol-containing) SDS-gels using anti-GFP, anti-Flag antibodies, and Streptavidin-HRP as a probe.

In Vivo Ubiquitination

293T cells were transfected with the construct encoding c-MET-C-GFPSpark. Cells were stimulated with either HGF WT or HGF 4Cys-4Ala as indicated following serum starvation. Cells were collected at indicated time points and washed with ice-cold PBS, lysed in ice-cold buffer containing 10 mM Tris-HCl (pH 8), 150 mM NaCl, 0.1% SDS, 20 mM NEM (N-ethylmaleimide) supplemented with protease and phosphatase inhibitors tablets (Thermo Fisher Scientific). The lysates were cleared by centrifugation at 10,000×g at 4° C. for 20 min, followed by preclearance using protein G-beads (protein G from Thermo Fisher). 500 μg of each precleared lysate was incubated with anti-MET antibody and protein-G beads for overnight at 4° C. with rotation. The samples were then washed three times with the lysis buffer and eluted with SDS-gel loading buffer (with reducing agent added). Proteins were resolved by SDS-PAGE and immunoblotted with the indicated antibodies.

Cell Proliferation Assay

3-(4, 5-dimethylthiazol-2-yl)-2, 5-diphenyl tetrazolium bromide (MTT) assay was used to determine cell viability. HEK293T and U-87-MG cells were plated in 96-well plates with 500 cells per well in triplicate in serum-free medium for 24 h prior to HGF stimulations. 100 ng mL-1 HGF WT, HGF 4Cys-4Ala, or both were added to the cells and incubated for 24 h prior to the addition of MTT solution (Abcam, Inc #ab211091) and cell viability was measured according to the manufacturer's instruction. The assays were performed in triplicate and the experiment was repeated three times. Data were expressed as the mean±SD. Statistical analyses were performed. *P<0.05 was considered to indicate a statistically significant difference.

Pathogen Infection and Analysis

Four-week-old Arabidopsis leaves were detached and inoculated with 5 μl Botrytis cinerea conidiospore suspension (5×105 spores ml-1 in potato dextrose broth) as previously described to analyze the disease symptoms (Ingle and Roden, 2014). Leaf lesions were pictured and measured using Image J software. For each genotype, at least 20 independent values were used for statistical analysis.

Protoplast Isolation and Transfection

Arabidopsis mesophyll protoplasts were isolated and transfected as described previously (Yoo et al., 2007). Protoplasts were isolated from fully expanded leaves from 3-4-wk-old Col-0 plants. For Cycloheximide and MG132 assay 20 μg of SpG-type LecRLK DNA were transfected into 200 μl 400 of protoplasts. After 18 h incubation, protoplasts were treated with MG-132 and Cycloheximide as indicated and were collected accordingly. Total proteins were extracted using protein extraction buffer for western blot analysis.

Plant RNA Extraction and qRT-PCR

Three-week-old leaf tissue was collected for RNA extractions using the Spectrum Plant Total RNA Kit (Sigma) and cDNA synthesized using the Superscript III Reverse Transcriptase with oligo-d(T) primers (Thermo Fisher Scientific). qRT-PCR was performed using the PowerUP SYBR Green Master Mix (Thermo Fisher Scientific) and relative expression for each gene was determined using 2ΔΔCt method with GAPDH as an internal control.

Phytohormone Analysis

Arabidopsis thaliana using electrospray ionization-high-performance liquid chromatography tandem mass spectrometry (ESI-HPLC-MS/MS). The plant hormones include jasmonic acid (JA), jasmonoyl-L-isoleucine (JA-ILE), abscisic acid (ABA), and salicylic acid (SA).

Preparation of Standard Solution of Plant Hormones

1) 2 μL of 500 mL standard reserve solution for each hormone was added to 986 μL methanol in a 1.5 mL centrifuge tube. The solution was mixed well and prepared as the stock solution with a final concentration of 1 mL. 2 μL of 500 mL internal standard reserve solution for each hormone was added to 996 μL methanol in a 1.5 mL centrifuge tube. The solution was mixed well and prepared as the stock solution with a final concentration of 1 μg/mL. Then, 989.9 μL, 989.8 μL, 989.5 μL, 988 μL, 985 μL, 970 μL, 940 μL, 790 μL methanol was added into 1.5 mL centrifuge tubes, and the stock solution prepared in step (1) was added into the methanol solution in sequence of 0.1 μL, 0.2 μL, 0.5 μL, 2 μL, 5 μL, 20 μL, 50 μL, 200 μL, respectively. 10 μL of the internal standard in step (2) was added to each tube to make standard solution with final concentration 0.1 ng/mL, 0.2 ng/mL, 0.5 ng/mL, 2 ng/mL, 5 ng/mL, 20 ng/mL, 50 ng/mL, 200 ng/mL with 10 ng/mL internal standard.

Steps for Phytohormones Extraction

1) About 1 g sample was weighed, grinded, and added into 10 times volume of acetonitrile solution with 4 μL internal standard stock solution.

2) The sample solution was briefly vortexed and kept at 4° C. overnight.

3) Following centrifugation for 5 min at 12,000 g at 4° C., the precipitate was extracted once again using 5 times volume of acetonitrile solution.

4) 35 mg C18 packing was added and mixed vigorously for 30 s, centrifuged at 10,000 g for 5 min, and the supernatant was saved.

5) The supernatant was dried by nitrogen and then dissolved in 400 μL methanol.

6) After filtration through a membrane filter (0.22 μm), 2 μL extract was injected onto a C18 column mounted on an analytical HPLC system (AGLIENT) equipped with AB SCIEX-6500 Qtrap MS/MS.

Western Blotting and Immunoprecipitation

Human Cell Lines: For in vivo stimulation experiments, cells were grown for 36 h and then stimulated with HGF WT and HGF 4Cys-4Ala where indicated (100 ng mL-1), washed with PBS, and lysed. Briefly, cell extracts were generated on ice in EBC buffer, 50 mM Tris (pH 8.0), 120 mM NaCl, 0.5% NP40, 1 mM DTT, and protease and phosphatase inhibitors tablets (Thermo Fisher Scientific). Extracted proteins were quantified using the Pierce™ BCA Protein assay kit (Thermo Fisher). Proteins were separated by SDS acrylamide gel electrophoresis and transferred to IMMOBILON-FL 26 PVDF membrane (Millipore) probed with the indicated antibodies and visualized either by chemiluminescence (according to the manufacturer's instructions) or using a LiCor Odyssey infrared imaging system. For immunoprecipitation, endogenous c-MET was immunoprecipitated on c-MET antibody-bound beads (Dynabeads Protein G from Thermo Fisher) and Flag-tagged HGF WT and HGF 4Cys-4Ala were in-vitro translated (TNT quick coupled Transcription/Translation system, Promega) and were incubated with the bead bound c-MET for 4 h at 4° C. Beads were then washed and proteins resolved by SDS-PAGE and analyzed by western blotting as described herein.

Plants: FLAG tagged SpG-type LecRLK-1 and SpG-type LecRLK-1R and GFP-tagged SpG-type LecRLK-1 and SpG-type LecRLK-1R were expressed where indicated in 293T cells. Cell extracts were generated in EBC buffer, 50 mM Tris (pH 8.0), 120 mM NaCl, 0.5% NP40, 1 mM DTT, and protease and phosphatase inhibitors tablets (Thermo Fisher Scientific). For protein phosphorylation, GFP tagged SpG-type LecRLK-1, and SpG-type LecRLK-1R were expressed in Arabidopsis. Protein extracts were generated using Sigma plant protein extraction kit (PE0230) from the transgenic plants. The isolated proteins were probed with GFP and pSerine antibody subsequently. For immunoprecipitation, equal amounts of cell lysates were incubated with the indicated antibodies conjugated to protein G beads (Invitrogen) (15 μl per IP, Thermo Scientific) overnight at 4° C. The beads were then washed with EBC buffer including inhibitors. Immunoprecipitation samples or equal amount of whole-cell lysates were resolved by SDS-PAGE, transferred to PVDF membranes (Millipore) probed with the indicated antibodies, and visualized with the LiCor Odyssey infrared imaging system.

Quantitative Real-Time PCR

Total RNA was extracted from glioblastoma U-87 MG and HEK293T cell line 24 h post HGF stimulation using TRIzol reagent (Invitrogen), and reverse transcription was performed using the SuperScript II RT kit (Integrative DNA Technologies) with total RNA (1 μg) according to the manufacturer's instructions. The c-MET and MMP9 mRNA expression levels were detected by conventional RT-PCR with Taq DNA Polymerase, Recombinant (Invitrogen, no. 10342-020). Glyceraldehyde-3-phosphate dehydrogenase (GAPDH) was used as the internal control. The specific primers for c-MET, MMP9 and GAPDH were designed with Primer Premier software. The primers used were c-MET, forward: 5′-TTAAAGGAGACCTCACCATGTAATC-3′ (SEQ ID NO: 6) and reverse: 5′-CCTGATCGAGAAACCACAACCT-3′ (SEQ ID NO: 7); MMP9, forward: 5′-GATCCAAAACTACTCGGAAGACTTG-3′ (SEQ ID NO: 8) and reverse: 5′-GAAGGCGCGGGCAAA-3′ (SEQ ID NO: 9) and GAPDH, forward: 5′-TTGCCATCAATGACCCCTTCA-3′ (SEQ ID NO: 10) and reverse: 5′-CGCCCCACTTGATTTTGGA-3′ (SEQ ID NO: 11). The PCR reaction was performed according to the manufacturer's instructions. The PCR conditions were as follows: amplification reaction protocol was performed for 35 cycles consisting of 30 s at 94° C. (denaturation), annealing 30 s at 45° C. and extension 30 s at 72° C.

Cloning, Heterologous Protein Expression, Purification, and Size Exclusion Chromatography

The PAN-like domain of SpG-type LecRLK-1 and SpG-type LecRLK-1R (amino acids 340-448) were amplified from plasmid templates pGWb402Omega-SpG-type LecRLK-1 and pGWb402Omega-SpG-type LecRLK-1R, respectively, using the following primers: AA340F (SEQ ID NO: 12) and RLK7_AA448R 5′-ACAAGAAAGCTGGGTCCTACACTTTTAAGAATGAAGT-3′ (SEQ ID NO: 13). Likewise, the PAN domain of SpG-type LecRLK-2 and SpG-type LecRLK-2R (amino acids 322-400) was amplified from plasmid templates pGWb402Omega-SpG-type LecRLK-2 and pGWb402Omega-SpG-type LecRLK-2R, respectively, using the following primers:

RLK5_AA322F

(SEQ ID NO: 14)

5′-AACTTGTACTTTCAAGGCGCTCTGAATTGTGATTCC-3′
and
RLK5_AA400R

(SEQ ID NO: 15)

5′-ACAAGAAAGCTGGGTCCTAAGATATCGAGCTTAAAGA-3′.

To create Gateway entry clones, attB-PCR products were generated using two-step adapter PCR (Prabhakar et al., 2020). The resulting expression constructs encode fusion proteins comprised of an NH2-terminal signal sequence, an 8×His tag, an AviTag recognition 482 site, the “superfolder” GFP (sfGFP) coding region, the recognition sequence of the tobacco etch virus (TEV) protease, followed by residues 322-400 of SpG-type LecRLK-2 (or the SpG-type LecRLK-2R variant) or residues 340-448 of SpG-type LecRLK-1 (or the SpG-type LecRLK-1R variant). Recombinant expression was accomplished by transient transfection of HEK293 cells (FreeStyle 293-F cells, ThermoFisher) at a 1L scale. Soluble secreted fusion proteins were purified from the culture media using HisTrap HP columns (Cytiva, USA) with an AKTA Pure 25L protein purification system (Cytiva, USA) as described previously (Prabhakar et al., 2020), and quantified using GFP fluorescence. The eluted fractions (more than 90% pure), identified by measuring GFP fluorescence. For high-resolution preparative gel filtration chromatography, 5 mL of purified, concentrated protein was injected onto a HiLoad 16/600 GL, Superdex 75 pg column (Cytiva, USA) pre-equilibrated with 50 mM HEPES, 400 mM NaCl using an ÄKTA Pure 25L protein purification system (Cytiva, USA). 5 mL fractions were collected based on absorbance at 280 nm with 5 mAU as a cutoff. All protein purification steps were carried out at 4° C.

RNA-Seq Experiment

Human Cell Line: Total RNA was extracted from the HEK293T cell line 24 h post HGF stimulation using TRIzol reagent (Invitrogen) according to the manufacturer's instructions. Quantification and quality control of isolated RNA was performed by measuring absorbance at 260 and 280 nm on a NANODROP ONEC spectrophotometer (Thermo Scientific, USA). The RNA-seq run was performed with four biological replicates. Library prep and sequencing was performed by BGI using the DNBSEQ-G400 platform which generated 100 bp paired-end reads. The raw RNA-seq reads have been deposited at NCBI under BioProject ID PRJNA718097. Clean reads were aligned to the human reference genome GRCh38. Reads were mapped with bowtie2 v2.2.559. Expression levels for RNAs were calculated using fragments per kilobase per million reads (FPKM) values with RSEM v1.2.860. Differential expression analysis was performed with DESeq2 and genes with an adjusted p-value less than 0.05 were considered differentially expressed.

Plants: RNA was extracted from transgenic tobacco and Arabidopsis leaves using the Sigma plant RNA extraction kit. Quantification and quality control of isolated RNA was performed by measuring absorbance at 260 nm and 280 nm on a NANODROP ONEC spectrophotometer (Thermo Scientific, USA). mRNA molecules were purified from total RNA using oligo(dT)-attached magnetic beads. Following that, mRNA molecules were fragmented into small pieces using fragmentation reagent after reaction a certain period in proper temperature. First-strand cDNA was generated using random hexamer-primed reverse transcription, followed by a second-strand cDNA synthesis. The synthesized cDNA was subjected to end-repair and then was 3′ adenylated. Adapters were ligated to the ends of these 3′ adenylated cDNA fragments. This process was to amplify the cDNA fragments with adapters from previous step. PCR products were purified with Ampure XP Beads (AGENCOURT) and dissolved in EB solution. Library was validated on the Agilent Technologies 2100 bioanalyzer. The double stranded PCR products were heat denatured and circularized by the splint oligo sequence. The single strand circle DNA (ssCir DNA) were formatted as the final library. The library was amplified with phi29 to make DNA nanoball (DNB). The DNBs were load into the patterned nanoarray and 150 paired-end 513 reads were generated in the way of sequenced by synthesis. The RNA-seq run was performed with four biological replicates. The raw RNA-seq reads have been deposited at NCBI under BioProject ID PRJNA739633. Clean RNA-Seq reads were mapped to the TAIR10 A. thaliana reference genome with bowtie2 v2.2.5 (Langmead and Salzberg, 2012). Expression level as Fragments per kilobase per million reads (FPKM) were generated with RSEM v1.2.8 (Li and Dewey, 2011). Differential expression analysis was performed with DESeq2, genes with an adjusted p-value less than 0.05 were considered differentially expressed (Love et al., 2014).

PAN Domain Alignment

PAN domains in SpG-type LecRLK-1 (SapurV1A.0918s0020), SpG-type LecRLK-2 (SapurV1A.0037s0270), Potri.T022200, SpG-typeV1A.1175s0020 (SapurV1A.1175s0020), SpG526 typeV1A.1316s0010 (SapurV1A.1316s0010), and AT1G61380 were identified with InterProScan 5 (Jones et al., 2014). The PAN domain region was extracted from the full-length protein and aligned with MAFFT linsi (Katoh and Standley, 2013). The alignment was visualized with Geneious Prime (Kearse et al., 2012).

Measurement of ROS Generation

Reactive oxygen species released by leaf tissue was determined by hydrogen peroxide-dependent luminescence of luminol. Leaf discs of 5-mm diameter from 4-week-old plants were punched and floated overnight in darkness in 96-well plates on 100111 distilled water. Horseradish peroxidase (1011 g/mL) and luminol (100 μm) from Sigma-Aldrich was added for elicitation and ROS detection. Luminescence was measured directly after addition of 50011M elicitor peptide Flg22 in plate reader Biotek Synergie2 for specified timepoints. For the H2DCFDA experiment, protocol was adapted from a previous report (Leshem et al., 2006). Briefly, 10-d-old seedlings, acclimated for 48 h in liquid MS medium, were incubated in 10 μm H2DCFDA (Sigma-Aldrich) in phosphate buffered saline solution for 30 min and washed twice for 5 min in phosphate-buffered saline solution. Chlorophyll autofluorescence and oxidized H2DCFDA were visualized using a Zeiss confocal microscope. H2DCFDA fluorescence was detected at 535 nm and chlorophyll at 650 nm and over.

Molecular Dynamics Simulations

MD simulations were initiated from the top five models generated with ColabFold27,62 of the WT and 4Cys-4Ala mutant PAN domain. The program tleap from AmberTools2063 was used to prepare the parameter and coordinate files for each structure. The ff14SB force field 64 and TIP3P water model 65 were used to describe the protein and solvent, respectively. Energy minimization was performed using sander from AmberTools20. At least a 12 Å solvent buffer between the protein and the periodic images. Sodium and chloride ions were added to neutralize charge and maintain a 0.10 M ion concentration. The simulations were performed with OpenMM version 7.5.166) on the Cuda platform (version 11.0.3) using Python 3.8.0. ParmEd was used to incorporate the force field parameters into the OpenMIVI platform 67. The Langevin integrator and Monte Carlo barostat were used to maintain the systems at 300 K and 1 bar, respectively. Direct non-bonded interactions were calculated up to a 12 Å distance cutoff. All bonds involving hydrogen atom were constrained to their equilibrium values. The particle mesh Ewald method was used to compute long-range Coulombic interactions. A 2 fs integration time step was used with energies and positions written every 2 ps.

Simulation Analysis

Analyses of MD trajectories were performed using Python 3.8.0 and the MDAnalysis version 1.0.169,70. Matplotlib was used to plot the data.

Statistical Analysis

Statistical analyses were performed on individual experiments, as indicated, with the GraphPad Prism Software using an unpaired t-test. Sample sizes and specific tests are indicated in the figure legends. A p-value of <0.05 was considered significant.

### SEQUENCES

Table 1 below describes sequences disclosed and used throughout the disclosure. These sequences are also included in the aforementioned electronic sequence listing (41946_5064_01_SequenceListing.xml of 61,440 bytes, created on May 24, 2023) incorporated by reference in its entirety.

TABLE 1

| SEQ ID NO: | Description |
|---|---|
| 1 | PAN Domain consensus sequence |
| 2 | Human HGF PAN Domain |
| 3 | PAN Domain consensus sequence |
| 4 | PAN Domain consensus sequence |
| 5 | PAN Domain consensus sequence |
| 6 | C-MET Forward Primer |
| 7 | c-MET Reverse Primer |
| 8 | MMP9 Forward Primer |
| 9 | MMP9 Reverse Primer |
| 10 | GAPDH Forward Primer |
| 11 | GAPDH Reverse Primer |
| 12 | AA340F Primer |
| 13 | RLK7_AA448R Primer |
| 14 | RLK5_AA322F Primer |
| 15 | RLK5_AA400R Primer |
| 16 | C. elegans LET-653 |
| 17 | D. melanogaster NOMP A |
| 18 | Danio rerio ADGRV1 |
| 19 | Rattus norvegicus PLG |
| 20 | B. taurus PLG |
| 21 | M. musculus HGF |
| 22 | H. sapiens HGF |
| 23 | C. lupus HGF |
| 24 | F. catus HGF |
| 25 | Z. mays PK1 |
| 26 | P. patens PHYPA_026294 |
| 27 | A. thaliana SD16 |
| 28 | O. sativa H0315F07.2 |
| 29 | P. trichocarpa POPTR_001G409300 |
| 30 | H. sapiens HGF |
| 31 | H. sapiens HGF mutated |

1. A method for modulating protein function, the method comprising modifying a target protein by attaching a PAN domain or a functional fragment thereof to the target protein thereby promoting internalization and degradation of the target protein, wherein the target protein does not comprise a native PAN domain.

2. The method of claim 1, wherein the PAN domain comprises SEQ ID NO: 1.

3. The method of claim 1, wherein the functional fragment of the PAN domain comprises an amino acid sequence that is at least 90% identical to amino acids 45-80 of SEQ ID NO: 1.

4.-8. (canceled)

9. The method of claim 1, wherein the PAN domain comprises an amino acid sequence that is at least 90% identical to SEQ ID NO: 2.

10.-15. (canceled)

16. The method of claim 1, wherein the target protein is involved in the RAF/MEK/ERK signaling cascade.

17. The method of claim 16, wherein the target protein is selected from C-RAF, ERK, and MAPK.

18. The method of claim 1, wherein the target protein is involved in the IGF/Akt signaling pathway.

19. The method of claim 18, wherein the target protein is selected from Akt, mTOR, p53, and PARP.

20. The method of claim 1, wherein the target protein is Kif11 protein.

21. The method of claim 1, wherein the target protein is associated with a pathogen.

22. The method of claim 21, wherein the target protein is SylA.

23. The method of claim 21, wherein the target protein is SKP1.

24. A method for promoting the internalization of a target extracellular protein without a PAN domain, the method comprising:
attaching a PAN domain or a functional fragment thereof to a ligand that binds the target extracellular protein, and
bringing the ligand attached with the PAN domain into contact with the target extracellular protein to permit binding of the ligand to the target extracellular protein, thereby initiating the internalization of the target extracellular protein by a cell.

25. The method of claim 24, wherein the PAN domain comprises SEQ ID NO: 1.

26. The method of claim 24, wherein the functional fragment of the PAN domain comprises an amino acid sequence that is at least 90% identical to amino acids 45-80 of SEQ ID NO: 1.

27. The method of claim 24, wherein the PAN domain comprises an amino acid sequence that is at least 90% identical to amino acids SEQ ID NO: 2.

28. The method of claim 24, wherein the functional fragment of the PAN domain comprises an amino acid sequence that is 90% identical to amino acids 70-96 of SEQ ID NO 2.

29. The method of claim 24, wherein the target extracellular protein is selected from microbe-associated molecular patterns (MAMPs).

30. The method of claim 29, wherein the active PAN domain is attached to a PRR, thereby initiating ubiquitination of the PRR.

31. The method of claim 29, wherein the MAMPs are from bacteria or fungi.

32. The method of claim 31, wherein the fungi are selected from Paecilomyces tenuipes and Beauveria bassiana.

33. A method for inhibiting the internalization of a targeted extracellular protein wherein the targeted extracellular protein comprises a PAN domain, the method comprising inactivating the PAN domain of the targeted extracellular protein.

34. The method of claim 33, wherein the inactivating the PAN domain of the targeted extracellular protein comprises substitution of at least one of the four conserved cysteines of SEQ ID NO: 1.

35.-37. (canceled)

38. The method of claim 33, wherein the inactivating the PAN domain of the targeted extracellular protein comprises substituting at least one of the four conserved cysteines of SEQ ID NO: 2.

39.-41. (canceled)
